(19) United States
(12) Patent Application Publication
(10) Pub. No.: US 2023/0144449 A1
(43) Pub. Date: May 11, 2023

(54) TERMINAL FUNCTIONAL SIDE CHAIN-SUBSTITUTED DIKETOPYRROLOPYRROLE (DPP)-BASED TERPOLYMER AND PREPARATION METHOD AND USE THEREOF

(71) Applicant: Beijing Institute Of Graphic Communication, Beijing (CN)

(72) Inventors: Shengzhen LIU, Beijing (CN); Zhaohui YU, Beijing (CN); Lijuan LIANG, Beijing (CN); Yun WENG, Beijing (CN); Ti WU, Beijing (CN); Xiaohui HE, Beijing (CN); Xuran WANG, Beijing (CN); Sunhao GUO, Beijing (CN)

(21) Appl. No.: 18/048,965

(22) Filed: Oct. 24, 2022

(30) Foreign Application Priority Data
Oct. 28, 2021 (CN) 202111263260.4

(51) Int. Cl. C08G 75/06

(52) U.S. Cl. CPC C08G 75/06

(57) ABSTRACT

A terminal functional side chain-substituted diketopyrrolopyrrole (DPP)-based terpolymer and a preparation method and use thereof is described herein. The terpolymer has the following structural formula:

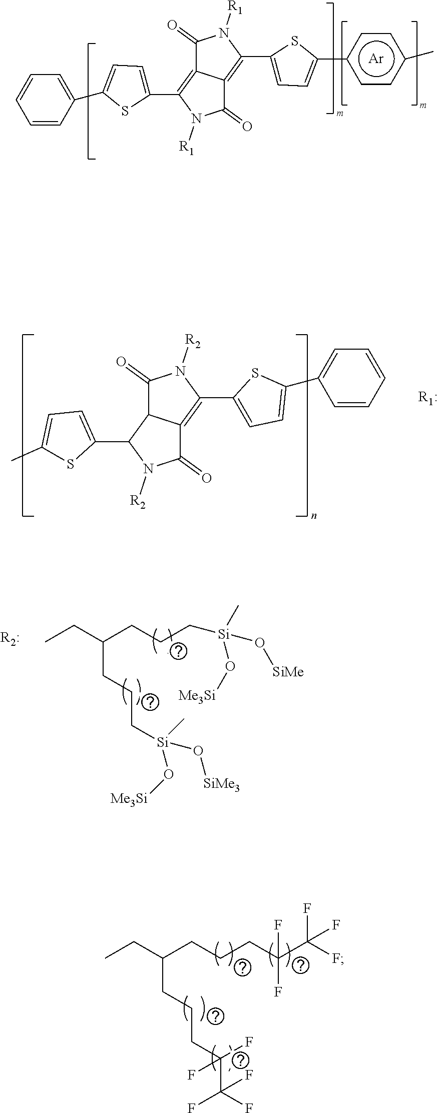

where R₁ is a terminal siloxy-substituted swallow-tailed chain with 22 to 52 carbon atoms in total, and t₁ and t₂ each are an integer of 1 to 18; R₂ is a semifluoroalkyl-substituted swallow-tailed chain with 12 to 60 carbon atoms in total and 10 to 46 fluorine atoms in total, t₃ and t₄ each are an integer of 1 to 16, and t₅ and t₆ each are an integer of 1 to 10; and Ar is any one selected from the group consisting of aryl, heteroaryl, substituent-containing aryl, and substituent-containing heteroaryl, and m and n each are an integer of 5 to 100.

### CROSS REFERENCE TO RELATED APPLICATION

This patent application claims the benefit and priority of Chinese Patent Application No. 202111263260.4, filed on Oct. 28, 2021, the disclosure of which is incorporated by reference herein in its entirety as part of the present application.

### TECHNICAL FIELD

The present disclosure belongs to the technical field of polymers, in particular to a terminal functional side chain-substituted diketopyrrolopyrrole (DPP)-based terpolymer and a preparation method and use thereof.

### BACKGROUND ART

Compared with the preparation of inorganic field effect transistors, organic polymer-based field effect transistors can be prepared by printing and other methods, with much simpler processes and procedures. Therefore, large-area and large-scale continuous production can be easily realized, which is beneficial to reduce production costs and simplify processes. Polymer field effect transistors can be widely used in large-scale integrated circuits, active matrix displays, sensors, and electronic trademarks, and have become the focus and hotspot of research and investment at home and abroad.

Diketopyrrolopyrrole (DPP) molecules, due to a large π coplanar structure, strong electron withdrawing ability, simple and efficient synthesis, and easy modification by alkyl chains to improve solubility, have been widely concerned and studied by researchers. The polymer solubility-promoting flexible chains have a great influence on polymer morphology. Appropriate selection of the flexible chains can promote ordered stacking of polymer molecules and improve thin film forms, thereby enhancing transistor device performances. Variations in length, position, and bulkiness of the flexible chain each may affect the above properties and have been extensively studied. At present, in order to change the form of polymer molecules and improve applicability, functional side chains such as fluorine chains are also introduced into the polymers due to special properties. However, due to a strong interaction between the fluorine chains, fluorine chain-modified polymers have a poor solubility and are easy to precipitate in a solution, resulting in poor solution processability of the terminal fluorine chain-modified polymers.

Therefore, based on the above problems, it is necessary to propose a new DPP-based polymer to improve the solubility of the polymer and further enhance an application range.

### SUMMARY

In order to solve at least one of the technical problems existing in the prior art, the present disclosure provides a terminal functional side chain-substituted DPP-based terpolymer, a preparation method of the terminal functional side chain-substituted DPP-based terpolymer, and use of the terminal functional side chain-substituted DPP-based terpolymer.

The present disclosure provides a terminal functional side chain-substituted DPP-based terpolymer, having the following structural formula:

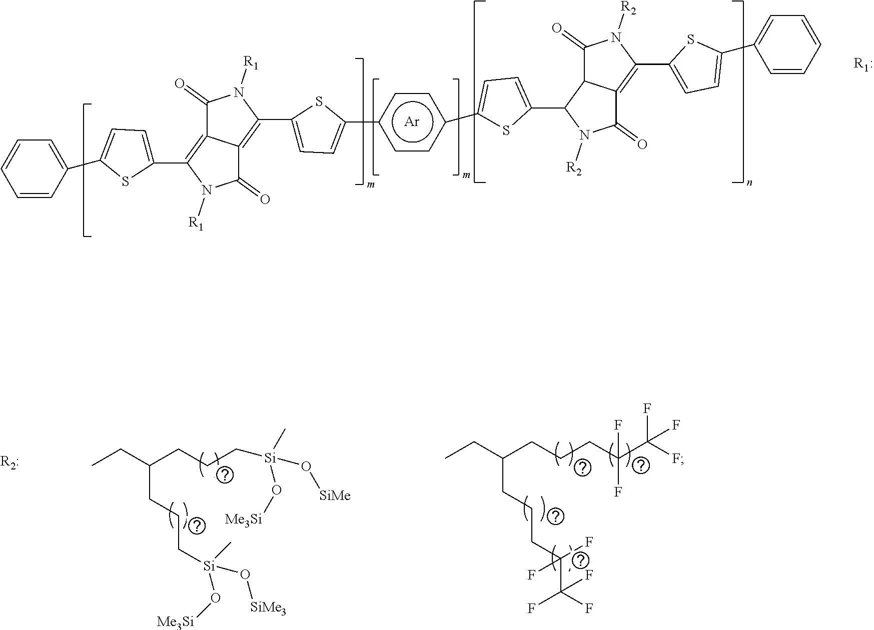

where

R₁ is a terminal siloxy-substituted swallow-tailed chain with 22 to 52 carbon atoms in total, and t₁ and t₂ each are an integer of 1 to 18;

R₂ is a semifluoroalkyl-substituted swallow-tailed chain with 12 to 60 carbon atoms in total and 10 to 46 fluorine atoms in total, t₃ and t₄ each are an integer of 1 to 16, and t₅ and t₆ each are an integer of 1 to 10; and

Ar is any one selected from the group consisting of aryl, heteroaryl, substituent-containing aryl, and substituent-containing heteroaryl, and m and n each are an integer of 5 to 100.

Optionally, in R₁, the branched alkyl with 22 to 52 carbon atoms in total may be any one selected from the group consisting of 2-pentylheptyl, 2-hexyloctyl, 2-heptylnonyl, 2-octyldecyl, 2-nonylundecyl, and 2-decyldodecyl; and/or,

in R₂, the branched alkyl with 12 to 60 carbon atoms in total may be any one selected from the group consisting of 4-undecylpentadecyl, 4-dodecylhexadecyl, and 4-tridecylheptadecyl, and the semifluoroalkyl-substituted fluorine chain with 10 to 46 fluorine atoms in total may be any one selected from the group consisting of nonafluorobutyl, heptafluoropropyl, and pentafluoroethyl; and/or,

in Ar, the aryl may be any one selected from the group consisting of monocyclic aryl, bicyclic aryl, and polycyclic aryl; and/or, the heteroaryl may be any one selected from the group consisting of monocyclic heteroaryl, bicyclic heteroaryl, and polycyclic heteroaryl; and/or, in the substituent-containing aryl and the substituent-containing heteroaryl, the substituent may be any one selected from the group consisting of C₁ to C₅₀ alkyl, C₁ to C₅₀ alkoxy, C₁ to C₅₀ alkylthio, a nitrile group, and a halogen atom, and there are 1 to 4 of the substituents.

Optionally, R₁ may be 10-ethyl-1,19-bis(1,1,1,3,5,5,5-heptamethyltrisiloxane)nonadecane; and/or,

R₂ may be 15-ethyl-1,1,1,2,2,3,3,4,4,26,26,27,27,28,28,29,29,29-octadecafluorononacosane; and/or,

Ar may be any one selected from the group consisting of:

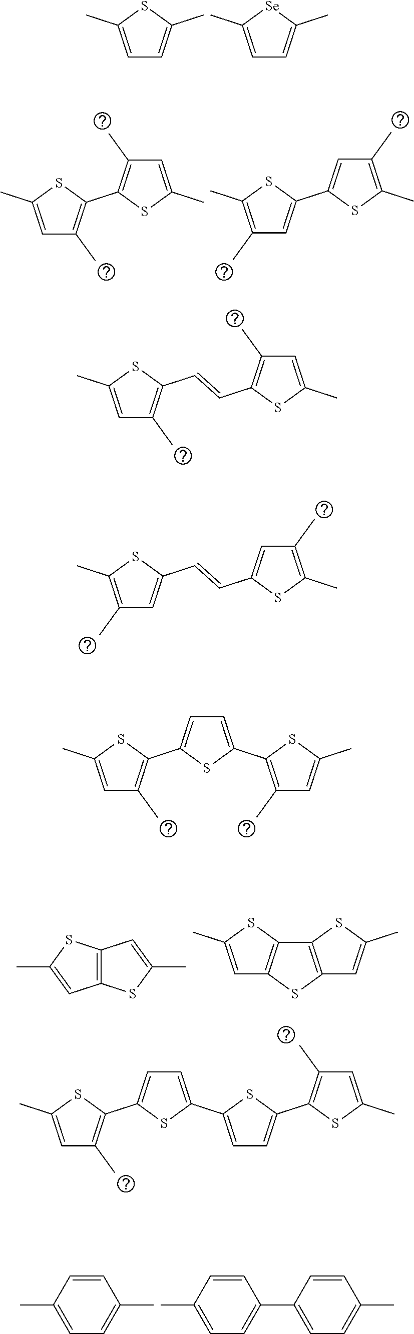

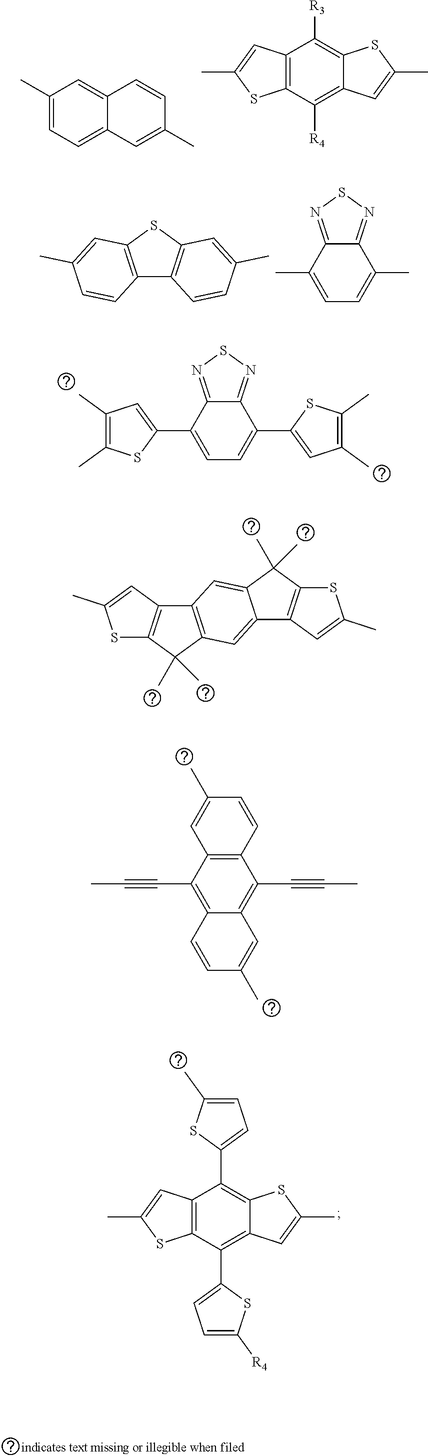

and

R₃ and R₄ each may be any one selected from the group consisting of hydrogen, C₁ to C₅₀ alkyl, C₁ to C₅₀ alkoxy, a nitrile group, and a halogen atom, and m and n each may be an integer of 5 to 50.

Optionally, heteroatoms in the monocyclic heteroaryl, the bicyclic heteroaryl, and the polycyclic heteroaryl each may be at least one selected from the group consisting of oxygen, sulfur, and selenium.

The present disclosure further provides a preparation method of the terminal functional side chain-substituted DPP-based terpolymer, including the following steps:

in the presence of an inert gas, a palladium catalyst, and a phosphine ligand, mixing the following monomers represented by M1, M2, and M3 in an organic solvent to obtain a reaction system, and conducting a reaction to obtain the terpolymer; where

M1 is

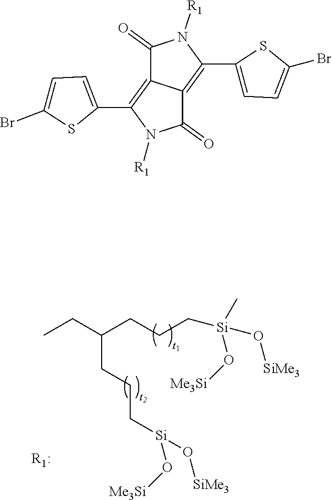

M2 is

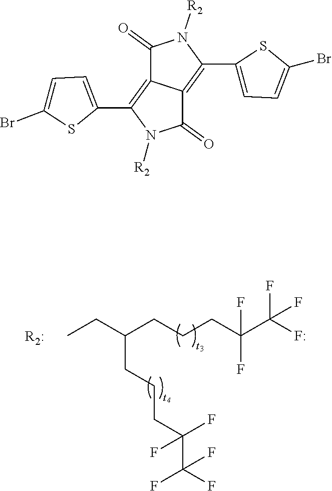

M3 is

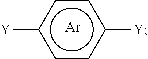

and Y is selected from the group consisting of a trialkyltin group and a borate group.

Optionally, the method may further include the following step after the reaction is completed: adding phenylboronic acid or bromobenzene to the reaction system to conduct a polymer end-capping treatment for 1 h to 24 h; where

the phenylboronic acid or the bromobenzene and the monomer represented by M1 have a molar dosage ratio of (10-100):1.

Optionally, the reaction may be conducted at 100° C. to 130° C. for 24 h to 72 h.

Optionally, the palladium catalyst, the phosphine ligand, and the monomer represented by M1 may have a molar dosage ratio of (0.01-0.05):(0.09-0.12):1; and/or,

the monomer represented by M1, the monomer represented by M2, and the monomer represented by M3 may have a molar dosage ratio of 1:(1-5.05):(2-6.05).

Optionally, the inert gas may be selected from the group consisting of nitrogen and argon; and/or,

the palladium catalyst may be at least one selected from the group consisting of tetrakis(triphenylphosphine)palladium, tris(tri-p-methylphenylphosphine)palladium, tris(dibenzylideneacetone)dipalladium, and [1,4-bis(diphenylphosphino)butane]palladium(II) dichloride; and/or,

the phosphine ligand may be at least one selected from the group consisting of triphenylphosphine, o-trimethylphosphine, tris(2-furyl)phosphine, and 2-(di-tert-butylphosphine)biphenyl; and/or,

the organic solvent may be at least one selected from the group consisting of toluene, chlorobenzene, and N,N-dimethylformamide; and/or,

the trialkyltin group may be selected from the group consisting of trimethyltin and tributyltin; and/or,

the borate group may be selected from the group consisting of 1,3,2-dioxaborolane-2-yl and 4,4,5,5-tetramethyl-1,2,3-dioxaborolane-2-yl.

The present disclosure further provides use of the terminal functional side chain-substituted DPP-based terpolymer in any one of an organic light-emitting diode, a field effect transistor, a flexible active matrix display, an organic radio frequency electronic trademark, an organic sensor/memory, an organic functional plastic, an electronic paper, and a solar cell.

In the present disclosure, functional side chains such as fluorine chains and siloxy branched chains are introduced at a terminal of the DPP, and the two group chains jointly modify the DPP to form DPP-based terpolymers with different substitutions. The siloxy chain has a desirable solubility to improve a solubility of a fluorine chain-modified polymer, such that the terpolymer has a desirable solubility. In addition, the DPP-based terpolymers with different substitutions each are a linear acceptor-donor-acceptor (A-D-A) conjugated molecule with an alternating ADA configuration and a rigid large π-planar structure, which are expected to fabricate high-mobility organic thin-film transistors (OTFTs) and other devices.

### BRIEF DESCRIPTION OF THE DRAWINGS

FIG. 1 shows a preparation flow chart of a DPP-based terpolymer in an example of the present disclosure;

FIG. 2 shows an ultraviolet-visible absorption spectrum of a DPP-based terpolymer in another example of the present disclosure in solution and solid thin film states;

FIG. 3 shows a cyclic voltammetry curve of a DPP-based terpolymer in another example of the present disclosure;

FIG. 4 shows a thermogravimetric analysis curve of a DPP-based terpolymer in another example of the present disclosure;

FIG. 5 shows a schematic structural diagram of an organic field effect transistor in which the DPP-based terpolymer in another example of the present disclosure is an organic active semiconductor layer;

FIG. 6 shows an output characteristic curve of the organic field effect transistor in which the DPP-based terpolymer in another example of the present disclosure is the organic active semiconductor layer; and

FIG. 7 shows a transfer characteristic curve of the organic field effect transistor in which the DPP-based terpolymer in another example of the present disclosure is the organic active semiconductor layer.

### DETAILED DESCRIPTION OF THE EMBODIMENTS

To enable those skilled in the art to better understand the technical solutions of the present disclosure, the present disclosure will be further described in detail below with reference to the accompanying drawings and specific embodiments. Apparently, the described embodiments are merely some rather than all of the embodiments of the present disclosure. All other embodiments obtained by those of ordinary skill in the art based on the described embodiments of the present disclosure without creative efforts should fall within the protection scope of the present disclosure.

At present, functional side chains such as fluorine chains are introduced into the DPP-based polymers. However, fluorine chain-modified polymers have a poor solubility and are easy to precipitate in a solution, resulting in poor solution processability of the terminal fluorine chain-modified polymers and the like. In the present disclosure, it is found that the siloxy branched chain-modified polymer has opposite properties to the fluorine chain-modified polymer, and has excellent solubility; the modified polymer is even soluble in low-polarity solvents such as n-hexane, that is, the siloxy branched chain has an obvious solubilization effect. Therefore, the terminal fluorine chain-modified polymer with excellent performance but poor solubility can be improved by the siloxy branched chain with an obvious solubilization effect, to form DPP-based small molecules and terpolymers co-modified by the siloxy chain, the fluorine chain, and the alkyl chain. This has not been reported in OTFTs.

The present disclosure provides a terminal functional side chain-substituted DPP-based terpolymer, having the following structural formula:

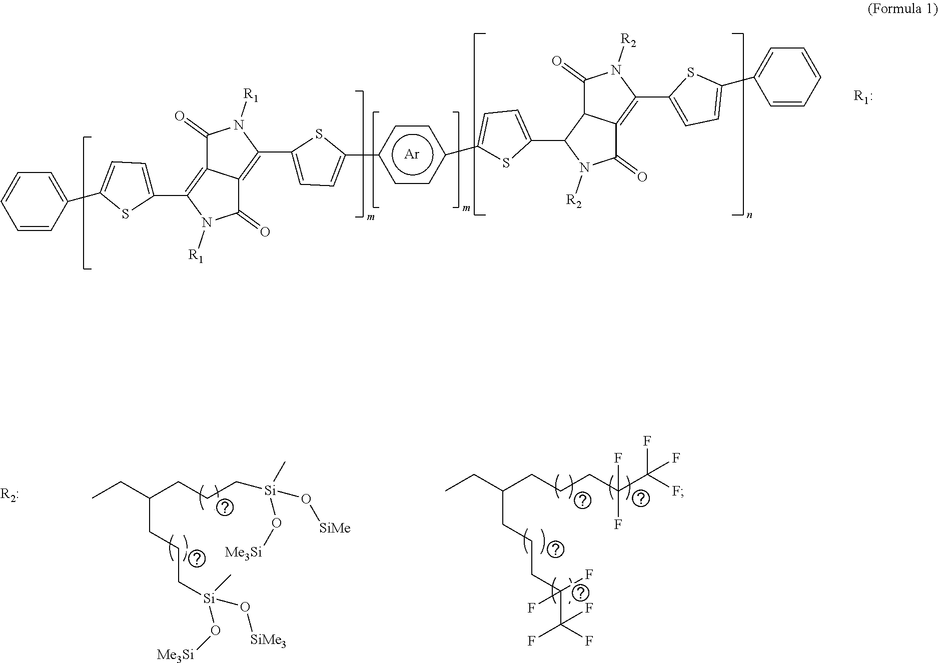

where

R₁ is a terminal siloxy-substituted swallow-tailed chain with 22 to 52 carbon atoms in total, and t₁ and t₂ each are an integer of 1 to 18; R₂ is a semifluoroalkyl-substituted swallow-tailed chain with 12 to 60 carbon atoms in total and 10 to 46 fluorine atoms in total, t₃ and t₄ each are an integer of 1 to 16, and t₅ and t₆ each are an integer of 1 to 10; and Ar is any one selected from the group consisting of aryl, heteroaryl, substituent-containing aryl, and substituent-containing heteroaryl, and m and n each are an integer of 5 to 100.

It should be noted that, in the present disclosure, there is no special limitation on specific groups selected for R₁, R₂ and Ar, and those skilled in the art can select the groups according to actual needs.

In some preferred examples, in R₁, t₁ and t₂ each are preferably an integer of 3 to 10; in R₂, t₃ and t₄ each are preferably an integer of 6 to 8, and t₅ and t₆ each are preferably an integer of 1 to 5; and in Ar, m and n each are preferably an integer of 5 to 50.

Further, in other preferred examples, in R₁, the branched alkyl with 22 to 52 carbon atoms in total is preferably any one selected from the group consisting of 2-pentylheptyl, 2-hexyloctyl, 2-heptylnonyl, 2-octyldecyl, 2-nonylundecyl, and 2-decyldodecyl. For example, R₁ is preferably 10-ethyl-1,19-bis(1,1,1,3,5,5,5-heptamethyltrisiloxane)nonadecane.

Further, in other preferred examples, in R₂, the branched alkyl with 12 to 60 carbon atoms in total is any one selected from the group consisting of 4-undecylpentadecyl, 4-dodecylhexadecyl, and 4-tridecylheptadecyl, and the semifluoroalkyl-substituted fluorine chain with 10 to 46 fluorine atoms in total is any one selected from the group consisting of nonafluorobutyl, heptafluoropropyl, and pentafluoroethyl. For example, R₂ is preferably 15-ethyl-1,1,1,2,2,3,3,4,4,26,26,27,27,28,28,29,29,29-octadecafluorononacosane.

Further, in other preferred examples, in Ar, the aryl is any one selected from the group consisting of monocyclic aryl, bicyclic aryl, and polycyclic aryl. The heteroaryl is any one selected from the group consisting of monocyclic heteroaryl, bicyclic heteroaryl, and polycyclic heteroaryl; and in the substituent-containing aryl and the substituent-containing heteroaryl, the substituent is any one selected from the group consisting of C₁ to C₅₀ alkyl, C₁ to C₅₀ alkoxy, C₁ to C₅₀ alkylthio, a nitrile group, and a halogen atom, and there are 1 to 4 of the substituents.

It should be noted that, heteroatoms in the monocyclic heteroaryl, the bicyclic heteroaryl, and the polycyclic heteroaryl each are at least one selected from the group consisting of oxygen, sulfur, and selenium, with no special limitation.

Further, in some other preferred examples, Ar is any one of the following unsubstituted or substituted groups selected from the group consisting of

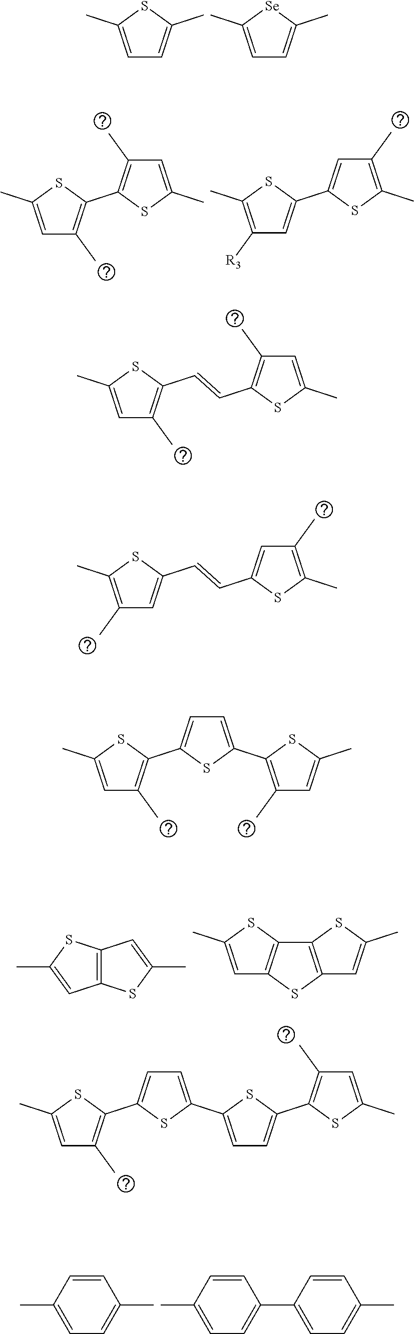

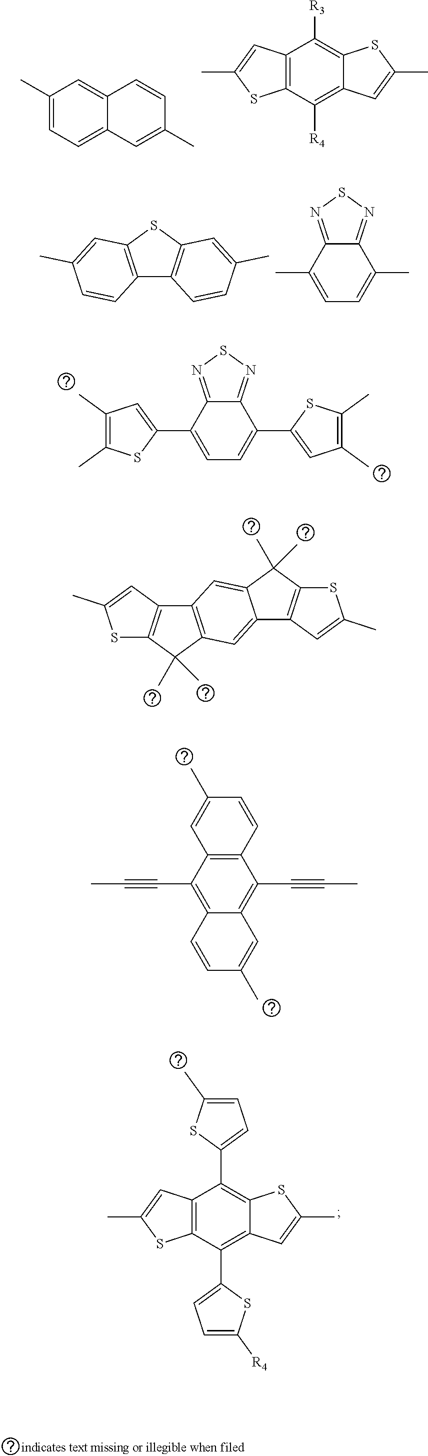

where

R₃ and R₄ each are any one selected from the group consisting of hydrogen, C₁ to C₅₀ alkyl, C₁ to C₅₀ alkoxy, a nitrile group, and a halogen atom, and m and n each are an integer of 5 to 50; and of course, in some other preferred examples, m and n each may preferably be an integer of 10 to 30.

It should be noted that, in the present disclosure, R₃ and R₄ groups may be the same or different; that is to say, R₃ and R₄ may be a same group or different groups, which are not specifically limited.

Exemplarily, in other preferred examples, Ar is preferably a selenophene group.

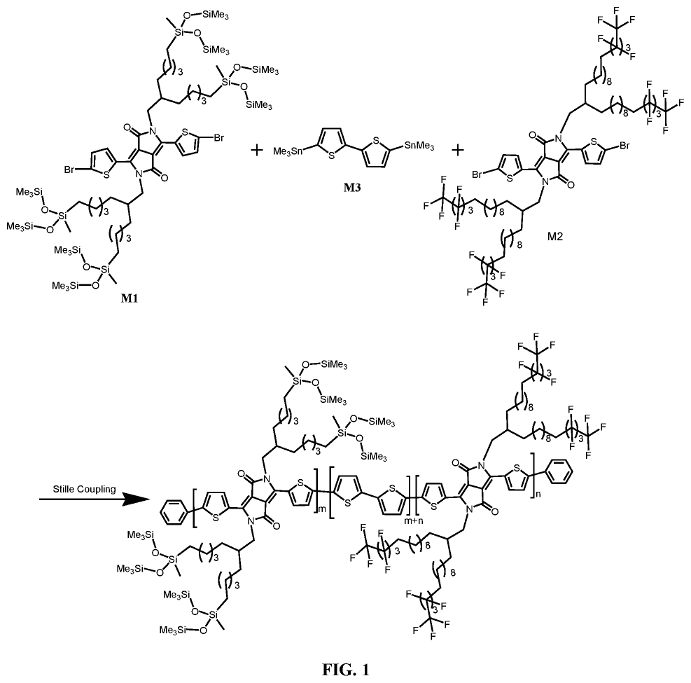

As shown in FIG. 1, the present disclosure further provides a preparation method of the terminal functional side chain-substituted DPP-based terpolymer, where the terpolymer has a structural formula referred to the above description, and details are not repeated here. In this example, the preparation method of the terpolymer includes the following steps, and a specific synthesis route is shown in FIG. 1:

in the presence of an inert gas, a palladium catalyst, and a phosphine ligand, mixing the following monomers represented by M1, M2, and M3 in an organic solvent to obtain a reaction system, and conducting a reaction to obtain the terpolymer; where

M1 is

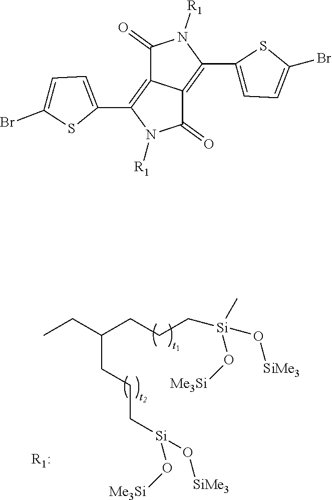

M2 is

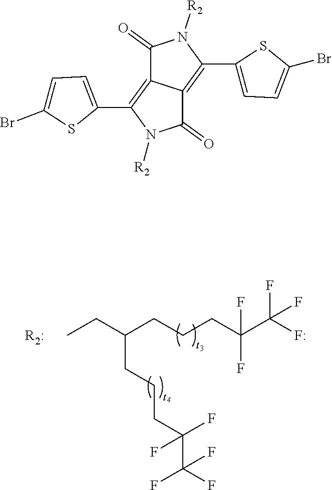

M3 is

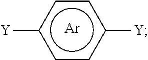

and Y is selected from the group consisting of a trialkyltin group and a borate group.

It should be noted that, R₁ is a terminal siloxy-substituted swallow-tailed chain with 22 to 52 carbon atoms in total, and t₁ and t₂ each are an integer of 1 to 18 (preferably 3 to 10); R₂ is a semifluoroalkyl-substituted swallow-tailed chain with 12 to 60 carbon atoms in total and 10 to 46 fluorine atoms in total, t₃ and t₄ each are an integer of 1 to 16 (preferably 6 to 8), and t₅ and t₆ each are an integer of 1 to 10 (preferably 1 to 5); and Ar is any one selected from the group consisting of aryl, heteroaryl, substituent-containing aryl, and substituent-containing heteroaryl, and m and n each are an integer of 5 to 100 (preferably 5 to 50).

Further, in other preferred examples, in R₁, the branched alkyl with 22 to 52 carbon atoms in total is preferably any one selected from the group consisting of 2-pentylheptyl, 2-hexyloctyl, 2-heptylnonyl, 2-octyldecyl, 2-nonylundecyl, and 2-decyldodecyl. For example, R₁ is preferably 10-ethyl-1,19-bis(1,1,1,3,5,5,5-heptamethyltrisiloxane)nonadecane.

Further, in other preferred examples, in R₂, the branched alkyl with 12 to 60 carbon atoms in total is any one selected from the group consisting of 4-undecylpentadecyl, 4-dodecylhexadecyl, and 4-tridecylheptadecyl, and the semifluoroalkyl-substituted fluorine chain with 10 to 46 fluorine atoms in total is any one selected from the group consisting of nonafluorobutyl, heptafluoropropyl, and pentafluoroethyl. For example, R₂ is preferably 15-ethyl-1,1,1,2,2,3,3,4,4,26,26,27,27,28,28,29,29,29-octadecafluorononacosane.

Further, in some other preferred examples, in Ar, there is any one of aryl, heteroaryl, substituent-containing aryl, and substituent-containing heteroaryl. For example, Ar is preferably a selenophene group. Moreover, the trialkyltin group is selected from the group consisting of trimethyltin and tributyltin, and the borate group is selected from the group consisting of 1,3,2-dioxaborolane-2-yl and 4,4,5,5-tetramethyl-1,2,3-dioxaborolane-2-yl. For example, Y is preferably a trimethyltin group.

Exemplarily, in some preferred examples, M3 is preferably 5,5-bistrimethylsilyl-2,2′-bithiophene.

Furthermore, the inert gas is selected from the group consisting of nitrogen and argon. The palladium catalyst is at least one selected from the group consisting of tetrakis(triphenylphosphine)palladium, tris(tri-p-methylphenylphosphine)palladium, tris(dibenzylideneacetone)dipalladium, and [1,4-bis(diphenylphosphino)butane]palladium(II) dichloride. The phosphine ligand is at least one selected from the group consisting of triphenylphosphine, o-trimethylphosphine, tris(2-furyl)phosphine, and 2-(di-tert-butylphosphine)biphenyl. The organic solvent is at least one selected from the group consisting of toluene, chlorobenzene, and N,N-dimethylformamide, with no special limitation.

In some preferred examples, the inert gas is the nitrogen, the palladium catalyst is the tris(dibenzylideneacetone)dipalladium, the phosphine ligand is the o-trimethylphosphine, and the organic solvent is preferably the chlorobenzene.

Further, in this example, the reaction is conducted at 100° C. to 130° C. for 24 h to 72 h; the palladium catalyst, the phosphine ligand, and the monomer represented by M1 have a molar dosage ratio of (0.01-0.05):(0.09-0.12):1; and the monomer represented by M1, the monomer represented by M2, and the monomer represented by M3 have a molar ratio range of 1:(1-5.05):(2-6.05).

Exemplarily, in this example, the reaction is conducted at 115° C. for 48 h; the tris(dibenzylideneacetone)dipalladium, the o-triphenylphosphine, and the monomer represented by M1 have a molar dosage ratio of 0.022:0.09:1; and the monomer represented by M1, the monomer represented by M2, and the monomer represented by M3 have a molar ratio range of 1:3:4.

It should be noted that, in this example, the method further includes the following step after the reaction is completed: adding phenylboronic acid or bromobenzene to the reaction system to conduct a polymer end-capping treatment for 1 h to 24 h; where the phenylboronic acid or the bromobenzene and the monomer represented by M1 have a molar dosage ratio of (10-100):1.

Exemplarily, in this embodiment, preferably the bromobenzene is added to the reaction system to conduct the polymer end-capping treatment for preferably 12 h. The phenylboronic acid or the bromobenzene and the monomer represented by M1 have a molar dosage ratio of preferably 50:1.

Further, in the example, the compound shown in M1 is prepared by the following method: under nitrogen protection in the dark, adding 100 mL of chloroform and 2,5-bis(siloxy-substituted alkyl)-3,6-bis(thiophen-2-yl)-2,5-dihydropyrrolo[3,4-c]pyrrole-1,4-dione (G), and adding N-bromosuccinimide (NBS) in batches under ice bath; stirring an obtained mixture at room temperature for 3 h; extracting with dichloromethane, combining organic phases, drying over magnesium sulfate, and spin-drying; purifying with dichloromethane/petroleum ether (1/1, V/V) by a chromatographic column to obtain the monomer M1 as a red-black liquid; where the G and the NBS have a molar ratio of 1:(2-2.4); and in some preferred examples, the molar ratio is preferably 1:2.2.

In addition, a synthetic route of the compound represented by M1 is as follows:

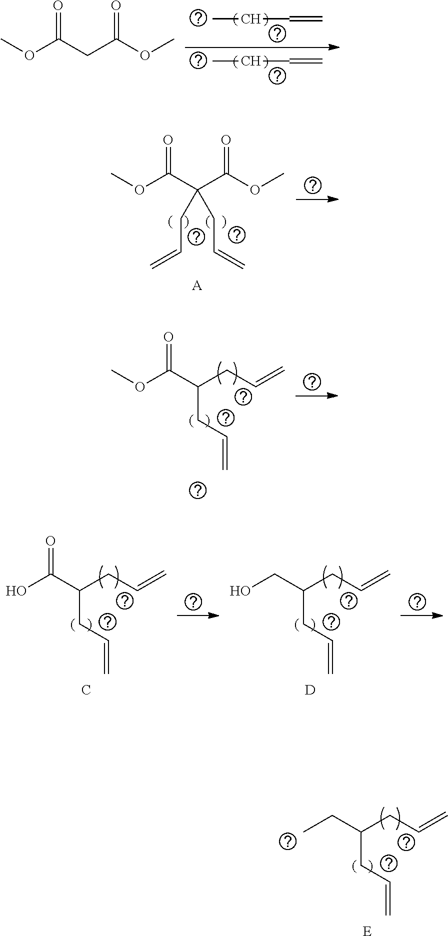

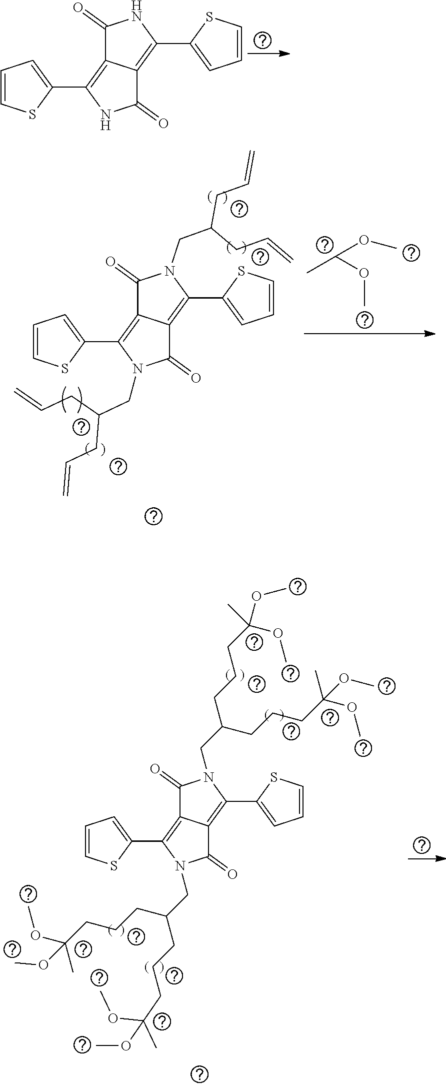

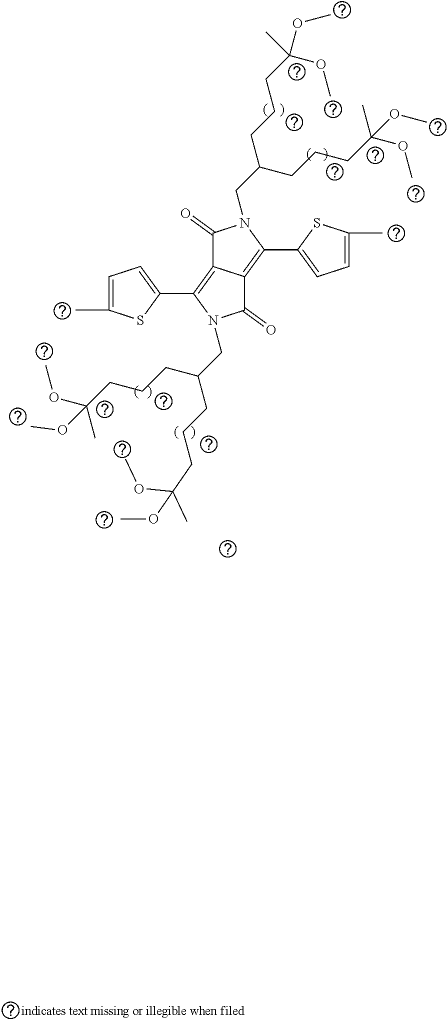

It should be noted that the compound represented by formula G in the above preparation process can be prepared according to the following method: under nitrogen protection, dissolving 2,5-bis(alkenyl)-3,6-bis(thiophen-2-yl)pyrrolo[3,4-c]pyrrole-1,4(2H,5H)-dione (F) in anhydrous toluene; adding 1,1,3,3,5,5,5-heptamethyltrisiloxane and a Karstedt catalyst (divinyltetramethylsiloxane complex, xylene, 2 wt) dropwise; stirring an obtained mixture at 70° C. overnight, spin-drying the solvent, and purifying by dichloromethane/petroleum ether (1/1, V/V) through a chromatographic column to obtain the product G as a reddish-black liquid; where the F and the 1,1,3,3,5,5,5-heptamethyltrisiloxane have a molar ratio of 1:(4-6); for example, the molar ratio may preferably be 1:4.8.

The compound represented by formula F can be prepared according to the following method: under nitrogen protection, in a 250 mL three-necked flask, adding 3,6-bis(thiophen-2-yl)-2,5-dihydropyrrolo[3,4-c]pyrrole-1,4-dione (DPP) and potassium carbonate in sequence, followed by adding a N,N-dimethylformamide (DMF) solvent and stirring; heating a resulting mixture to 110° C. and stirring for 1 h, adding iodoalkenyl-dovetailene (E), heating to 120° C. overnight; cooling to room temperature, removing the potassium carbonate by suction filtration, spin-drying, and purifying with dichloromethane/petroleum ether (1/1, V/V) by a chromatographic column to obtain the red powdery solid F; where the 3,6-bis(thiophen-2-yl)-2,5-dihydropyrrolo[3,4-c]pyrrole-1,4-dione, the potassium carbonate, and the E have a molar ratio of 1:(3-3.5):(2-2.4); and for example, the molar ratio may preferably be 1:3:2.2.

Further, the compound shown in formula E can be prepared according to the following method: adding alkenyl-dovetailenol (D) and dichloromethane and stirring in a single-necked flask in the dark; adding triphenylphosphine and imidazole in sequence, adding iodine in batches under ice bath, conducting a reaction overnight at room temperature, and subjecting a product to spin-drying, filtration, water washing, drying, and spin-drying to obtain a colorless liquid product E; where the D, the triphenylphosphine, the imidazole, and the iodine have a molar ratio of 1:(1.1-1.5):(1.1-1.5):(1.1-1.3); and for example, the molar ratio may preferably be 1:1.2:1.2:1.15.

Further, the compound shown formula D can be prepared according to the following method: under nitrogen protection, dissolving lithium aluminum hydride in anhydrous tetrahydrofuran by stirring, dissolving alkenyl-dovetail olefine acid (C) in anhydrous tetrahydrofuran by adding dropwise, and refluxing for 4 h; cooling a mixture to room temperature, slowly adding water, dissolving with 30% sulfuric acid, and extracting with ether; subjecting a product to washing with an aqueous sodium thiosulfate solution, washing with water, washing with a saline, drying, spin-drying, and purifying with dichloromethane/petroleum ether (2/1, V/V) through a chromatographic column to obtain the product D as a colorless liquid; where the C and the lithium aluminum hydride have a molar ratio of 1:(1-2); and for example, the molar ratio may preferably be 1:1.5.

Further, the compound shown formula C can be prepared according to the following method: dissolving alkenyl-alkenoic acid methyl ester (B) in ethanol, adding 1 M sodium hydroxide solution, and refluxing for 6 h; cooling a mixture to room temperature, adding 2 M hydrochloric acid solution, stirring for 30 min, and extracting with ethyl acetate; subjecting a product to washing with water, washing with a saline, drying, spin-drying, and purifying with ethyl acetate/petroleum ether (9/1, V/V) by a chromatographic column to obtain the colorless liquid product C; where the B and the sodium hydroxide (1 M) have a molar ratio of 1:(1-1.2); for example, the molar ratio may be preferably 1:1.

Further, the compound shown in formula B can be prepared according to the following method: adding alkenyl-dimethyl malonate (A), dimethyl sulfoxide, lithium chloride, and water successively to a single-necked bottle, and conducting a reflux reaction at 189° C. for 6 h; pouring a product into water, extracting with ether, followed drying, spin-drying, and purifying with dichloromethane/petroleum ether (1/2, V/V) through a chromatographic column to obtain the colorless liquid product B; where the A, the lithium chloride, and water have a molar ratio of 1:(2-2.3):(1-1.2); for example, the molar ratio may be preferably 1:2:1.1.

The compound shown in formula A can be prepared according to the following method: under nitrogen protection, adding sodium methoxide and dimethyl malonate to a 250 mL three-necked flask, adding t₁-bromoalkene dropwise, and refluxing a mixture at 65° C. for 6 h; spin-drying a product, pouring into water, extracting with ether, drying, and spin-drying to obtain an intermediate; adding the sodium methoxide to the intermediate, adding t₂-bromoalkene dropwise, and refluxing a mixture at 65° C. for 6 h; spin-drying a product, pouring into water, extracting with ether, drying, and spin-drying to obtain the colorless liquid product A; where the sodium methoxide, the dimethyl malonate, the t₁-bromoalkene, and the t₂-bromoalkene have a molar ratio of (1.3-1.5):1:(1-1.1):(1-1.1); for example, the molar ratio may be preferably 1.3:1:1.05:1.05.

Further, in the example, the compound shown in M2 is prepared by the following method: under nitrogen protection in the dark, adding 100 mL of chloroform and 2,5-bis(fluoro-substituted alkyl)-3,6-bis(thiophen-2-yl)-2,5-dihydropyrrolo[3,4-c]pyrrole-1,4-dione (E1) in a 250 mL three-necked flask; adding N-bromosuccinimide (NBS) in batches under an ice bath; stirring a mixture at room temperature for 3 h, extracting with dichloromethane, combining organic phases, drying over magnesium sulfate, spin-drying, and purifying with dichloromethane/petroleum ether (1/1, V/V) through a chromatographic column to obtain the red powder monomer M2; where the E1 and the NBS have a molar ratio of 1:(2-2.4); and for example, the molar ratio may be preferably 1:2.2.

In addition, a synthetic route of the compound represented by M2 is as follows:

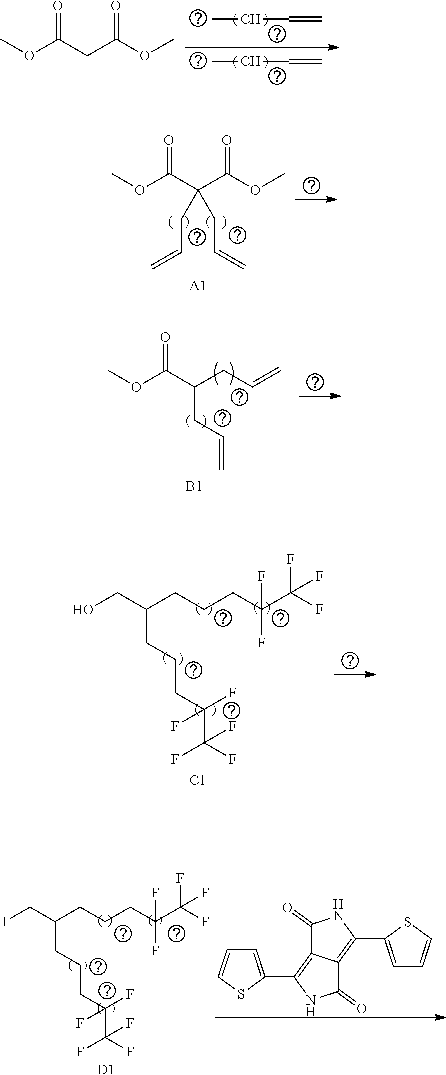

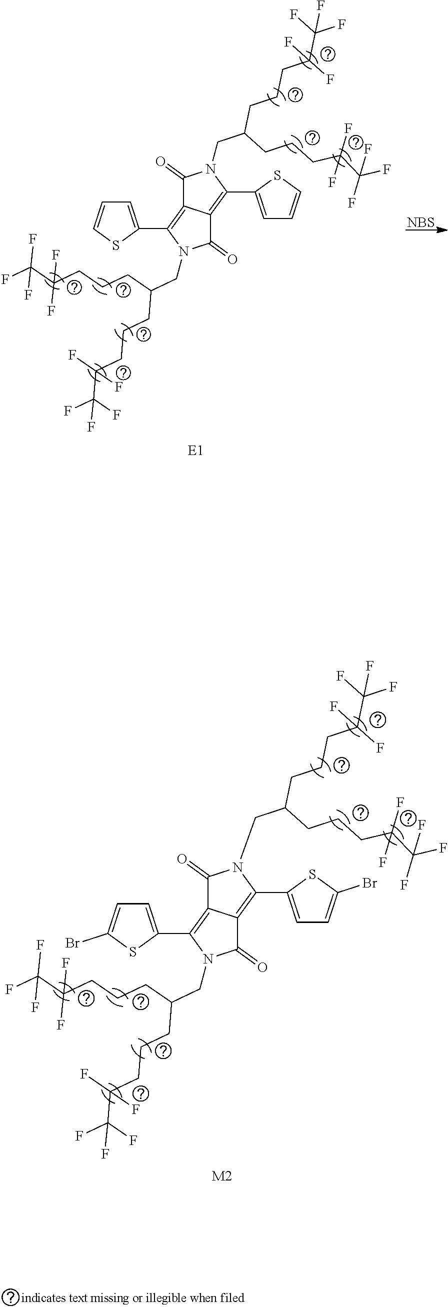

It should be noted that the compound represented by formula E1 in the above preparation process can be prepared according to the following method: under nitrogen protection, adding 3,6-bis(thiophen-2-yl)-2,5-dihydropyrrolo[3,4-c]pyrrole-1,4-dione and potassium carbonate sequentially to a 250 mL three-necked flask; adding a N,N-dimethylformamide (DMF) solvent and stirring, heating to 110° C. and stirring for 1 h; adding iodosemifluoroalkyl-substituted dovetail alkyl (D1), heating to 120° C. overnight; cooling a mixture to room temperature, removing the potassium carbonate by suction filtration, spin-drying, and purifying with dichloromethane/petroleum ether (1/1, V/V) by a chromatographic column to obtain the red powdery solid E1; where the 3,6-bis(thiophen-2-yl)-2,5-dihydropyrrolo[3,4-c]pyrrole-1,4-dione, the potassium carbonate, and the D1 have a molar ratio of 1:(3-3.5):(2-2.4); and the molar ratio may be preferably 1:3:2.2.

Further, the compound shown in formula D1 can be prepared according to the following method: adding semifluoroalkyl-substituted dovetail alcohol (C1) and dichloromethane and stirring in a single-necked flask in the dark, and adding triphenylphosphine and imidazole in sequence; adding iodine in batches under ice bath, conducting a reaction overnight at room temperature, and subjecting a product to spin-drying, filtration, water washing, drying, and spin-drying to obtain a colorless liquid product D1; where the C1, the triphenylphosphine, the imidazole, and the iodine have a molar ratio of 1:(1.1-1.5):(1.1-1.5):(1.1-1.3); and for example, the molar ratio may be preferably 1:1.2:1.2:1.15.

Further, the compound shown in formula C1 can be prepared according to the following method: adding alkenoic acid methyl ester (B1), water-free n-hexane, and perfluoroiodoalkane sequentially in a single-necked flask in the dark; under a liquid nitrogen environment, replacing nitrogen and heating to room temperature, repeating the above operations three times; cooling to 0° C., adding tetrakis(triphenylphosphine)palladium, conducting a reaction at room temperature for 48 h, filtering a product through a dry silica gel column, and spin-drying; under nitrogen conditions, adding lithium aluminum hydride and water-free ether to another single-necked bottle; adding the spin-dried filtrate obtained in the previous step dropwise to the ether, at a speed that the solution is refluxed, and conducting a reflux reaction for 6 h; cooling a product to room temperature, slowly adding water and 30% sulfuric acid, and extracting with ether; subjecting a product to washing with an aqueous solution of sodium thiosulfate, washing with water, washing with saline, drying, spin-drying, and purifying with dichloromethane/petroleum ether (2/1, V/V) through a chromatographic column to obtain the colorless liquid product C1; where the B1, the perfluoroiodoalkane, the tetrakis(triphenylphosphine)palladium, and the lithium aluminum hydride have a molar ratio of 1:(2-2.5):(0.02-0.06):(3-3.3); and for example, the molar ratio may be preferably 1:2.15:0.024:3.

Further, the compound shown in formula B1 can be prepared according to the following method: adding alkenyl-dimethyl malonate (A1), dimethyl sulfoxide, lithium chloride, and water successively to a single-necked bottle, and conducting a reflux reaction at 189° C. for 6 h; pouring a product into water, extracting with ether, followed drying, spin-drying, and purifying with dichloromethane/petroleum ether (1/2, V/V) through a chromatographic column to obtain the colorless liquid product B1; where the A1, the lithium chloride, and water have a molar ratio of 1:(2-2.3):(1-1.2); and for example, the molar ratio may be preferably 1:2:1.1.

The compound shown in above formula A1 can be prepared according to the following method: under nitrogen protection, adding sodium methoxide and dimethyl malonate to a 250 mL three-necked flask, and adding t₃-bromoalkene dropwise; refluxing at 65° C. for 6 h; spin-drying a product, pouring into water, extracting with ether, drying, and spin-drying to obtain an intermediate; adding the sodium methoxide to the intermediate, adding t₄-bromoalkene dropwise, and refluxing a mixture at 65° C. for 6 h; spin-drying a product, pouring into water, extracting with ether, drying, and spin-drying to obtain the colorless liquid product A; where the sodium methoxide, the dimethyl malonate, the t₃-bromoalkene, and the t₄-bromoalkene have a molar ratio of (1.3-1.5):1:(1-1.1):(1-1.1); and for example, the molar ratio may be preferably 1.3:1:1.05:1.05.

The present disclosure further provides use of the terminal functional side chain-substituted DPP-based terpolymer in any one of an organic light-emitting diode, a field effect transistor, a flexible active matrix display, an organic radio frequency electronic trademark, an organic sensor/memory, an organic functional plastic, an electronic paper, and a solar cell.

It should be noted that the compounds can be widely used as carrier transport compounds in electronic devices, such as semiconductor materials, and can be used as effective components of organic light-emitting diodes (OLEDs), field effect transistors (FETs) and solar cells.

The following will further illustrate the preparation method of the terminal functional side chain-substituted DPP-based terpolymer of the present disclosure in conjunction with specific examples:

S1, a compound shown in formula A was prepared according to the following method:

under nitrogen protection, in a 250 mL three-necked flask, 65 g (30% wt) of sodium methoxide and 16.55 g (125 mmol) of dimethyl malonate were added, 40 g (268 mmol) of 5-bromo-1-pentene (t₁=t₂=3) was dropwise added, and refluxed at 65° C. for 6 h; a product was spin-dried, poured into water, extracted with ether, dried, and spin-dried to obtain a colorless liquid product, dimethyl 2,2-bis(4-enyl-pentane)malonate (A), which was used in a next reaction directly.

S2, a compound shown in formula B was prepared according to the following method:

17 g (63.5 mmol) of the 2,2-bis(4-enyl-pentane)dimethyl malonate (A) obtained in step S1, 150 mL of dimethyl sulfoxide, 5.42 g of (127 mmol)) lithium chloride, and 1.2 g (65 mmol) of water were added to a single-necked flask successively, and a reaction was conducted at 189° C. by refluxing for 6 h; a product was poured into water, extracted with ether, dried, spin-dried, and purified with dichloromethane/petroleum ether (1/2, V/V) through a chromatographic column to obtain a colorless liquid product, 2-(4-enyl-pentane)heptyl-6-alkenoic acid methyl ester (B) 11.2 g, with a yield of 84%. Product structural characterization data was as follows:

Hydrogen NMR spectrum: ¹H NMR (600 MHz, CDCl₃): 5.815.77 (m, 2H), 5.034.95 (m, 4H), 3.69 (s, 3H), 2.382.36 (m, 1H), 2.07-2.04 (m, 4H), 1.65-1.60 (m, 2H), 1.50-1.45 (m, 2H), 1.401.35 (m, 4H).

Carbon NMR spectrum: ¹³C NMR (150 MHz, CDCl₃): 176.72, 138.41, 138.33, 114.72, 114.65, 114.42, 67.89, 51.32, 45.44, 45.38, 33.57, 31.86, 31.79, 26.68, 26.54.

S3, a compound shown in formula C was prepared according to the following method:

11.2 g (53 mmol) of the 2-(4-enyl-pentane)heptyl-6-alkenoic acid methyl ester (B) obtained in step S2 was dissolved in 100 mL of ethanol, 42 mL of 1 M sodium hydroxide solution was added, and refluxed for 6 h; a mixture was cooled to room temperature, 50 mL of 2 M hydrochloric acid solution was added, stirred for 30 min, and extracted with ethyl acetate; an obtained product was subjected to washing with water, washing with a saline, drying, spin-drying, and purifying with ethyl acetate/petroleum ether (9/1, V/V) by a chromatographic column to obtain 10.3 g of a colorless liquid product 2-(4-alkenyl-pentane)heptyl-6-alkenoic acid (C), with a yield of 95%. Product structural characterization data was as follows:

Hydrogen NMR spectrum: ¹H NMR (600 MHz, CDCl₃): 5.835.78 (m, 2H), 5.054.97 (m, 4H), 2.39 (s, 1H), 2.10-2.07 (m, 4H), 1.68-1.66 (m, 2H), 1.54-1.43 (m, 6H).

Carbon NMR spectrum: ¹³C NMR (150 MHz, CDCl₃): 182.24, 138.33, 114.75, 45.18, 33.55, 31.52, 26.55.

S4, a compound shown in formula D was prepared according to the following method:

under nitrogen protection, 3 g (79 mmol) of lithium aluminum hydride was dissolved in 50 mL of anhydrous tetrahydrofuran by stirring, and 10.3 g (52.5 mmol) of the 2-(4-alkenyl-pentane)heptyl-6-alkenoic acid (C) obtained in step S3 was added dropwise and dissolved in 30 mL of anhydrous tetrahydrofuran. A reflux reaction was conducted for 4 h; a product was cooled to room temperature, water was slowly added, dissolved with 30% sulfuric acid, and extracted with ether; an obtained product was subjected to washing with an aqueous solution of sodium thiosulfate, washing with water, washing with saline, drying, spin-drying, and purifying with dichloromethane/petroleum ether (2/1, V/V) through a chromatographic column to obtain 8.2 g of a colorless liquid product 2-(4-alkenyl-pentane)heptyl-6-enol (D), with a yield of 86%. Product structural characterization data was as follows:

Hydrogen NMR spectrum: ¹H NMR (600 MHz, CDCl₃): 5.855.80 (m, 2H), 5.044.95 (m, 4H), 3.56 (s, 2H), 2.092.05 (m, 4H), 1.441.31 (m, 9H).

Carbon NMR spectrum: ¹³C NMR (150 MHz, CDCl₃): 138.87, 114.42, 65.57, 40.46, 40.35, 40.23, 34.10, 30.51, 30.40, 30.28, 26.31, 26.21, 26.08.

S5, a compound shown in formula E was prepared according to the following method:

In the dark, 8.2 g (45 mmol) of the 2-(4-alkenyl-pentane) heptyl-6-enol (D) obtained in step S4 and 100 mL of dichloromethane were added to a single-necked bottle, and stirred; 14.15 g (54 mmol) of triphenylphosphine and 3.66 g (54 mmol) of imidazole were sequentially added, and 13.5 g (53 mmol) of iodine was added in batches under ice bath; a mixture was reacted overnight at room temperature; a product was spin-dried, filtered through a dry silica gel column with suction filtration, washed with water, dried, and spin-dried to obtain 10.5 g of a colorless liquid product, 6-(iodomethyl)undec-1,10-diene (E), with a yield of 80%.

S6, a compound shown in formula F was prepared according to the following method:

under nitrogen protection, 2.4 g (8 mmol) of 3,6-bis(thiophen-2-yl)-2,5-dihydropyrrolo[3,4-c]pyrrole-1,4-dione, 3.32 g (24 mmol) of potassium carbonate were added, and 150 mL of a N,N-dimethylformamide (DMF) solvent was added and stirred; a mixture was heated to 110° C. and stirred for 1 h, 7 g (24 mmol) of the 6-(iodomethyl)undec-1,10-diene (E) obtained in step S5 was added, and heated to 120° C. overnight; after cooling to room temperature, the potassium carbonate was removed by suction filtration, and a product was spin-dried, and purified with dichloromethane/petroleum ether (1/1, V/V) by a chromatographic column to obtain 1.76 g of a red powdery solid 2,5-bis(2-(pent-4-enyl)hept-6-enyl)-3,6-bis(thiophen-2-yl)pyrrolo[3,4-c]pyrrole-1,4(2H,5H)-dione (F), with a yield of 35%. Product structural characterization data was as follows:

Mass spectrum: MALDI-TOF: m/z 628.3.

Hydrogen NMR spectrum: ¹H NMR (600 MHz, CDCl₃): 8.86 (t, 2H), 7.65-7.64 (m, 2H), 7.30-7.28 (m, 2H), 5.795.74 (m, 4H), 4.974.90 (m, 8H), 4.05-4.04 (d, 4H), 2.011.98 (m, 10H), 1.461.33 (m, 16H).

Carbon NMR spectrum: ¹³C NMR (150 MHz, CDCl₃): 177.36, 161.74, 140.39, 138.67, 135.16, 130.48, 129.77, 128.43, 114.48, 108.03, 46.02, 37.54, 34.00, 30.58, 25.48.

S7, a compound shown in formula G was prepared according to the following method:

under nitrogen protection, 1.176 g (1.87 mmol) of the 2,5-bis(2-(pent-4-enyl)hept-6-enyl)-3,6-bis(thiophen-2-yl)pyrrolo[3,4-c]pyrrole-1,4(2H,5H)-dione (F) obtained in step S6 was dissolved in anhydrous toluene; 2 g (8.98 mmol) of 1,1,3,3,5,5,5-heptamethyltrisiloxane and a catalytic amount of a Karstedt catalyst (divinyltetramethylsiloxane complex, xylene, 2 wt) were added dropwise; an obtained mixture was stirred at 70° C. overnight, the solvent was spin-dried, and purified by dichloromethane/petroleum ether (1/1, V/V) through a chromatographic column to obtain 2 g of a red-black liquid product 2,5-bis(6-ethyl-1,11-bis(1,1,1,3,5,5,5-heptamethyltrisiloxane))-3,6-bis(thiophen-2-yl)pyrrolo[3,4-c]pyrrole-1,4(2H,5H)-dione (G), with a yield of 70%. Product structural characterization data was as follows:

Mass spectrum: MALDI-TOF: m/z 1517.6.

Hydrogen NMR spectrum: ¹H NMR (600 MHz, CDCl₃): 8.89-8.88 (m, 2H), 7.63-7.62 (m, 2H), 7.29-7.27 (m, 2H), 4.04-4.03 (d, 4H), 1.98-1.94 (t, 4H), 1.321.24 (m, 40H), 0.11-0.10 (m, 84H).

Carbon NMR spectrum: ¹³C NMR (150 MHz, CDCl₃): 161.74, 140.43, 135.18, 130.34, 129.85, 128.37, 107.97, 46.21, 37.95, 33.71, 31.10, 25.97, 23.08, 17.63, 2.05, 1.85, 1.65, 1.00.

S8, a compound shown in formula M1 was prepared according to the following method:

under nitrogen protection in the dark, adding 100 mL of chloroform and 1.52 g of the 2,5-bis(6-ethyl-1,11-bis(1,1,1,3,5,5,5-heptamethyltrisiloxane))-3,6-bis(thiophen-2-yl)pyrrolo[3,4-c]pyrrole-1,4(2H,5H)-dione (G) obtained in step S7 in a 250 mL three-necked flask; 374 mg (2.1 mmol) of N-bromosuccinimide (NBS) was added in batches under an ice bath; a mixture was stirred at room temperature for 3 h, extracted with dichloromethane, organic phases were combined, dried over magnesium sulfate, spin-dried, and purified with dichloromethane/petroleum ether (1/1, V/V) through a chromatographic column to obtain 0.587 g of a red-black liquid product (M1), with a yield of 35%. Product structural characterization data was as follows:

Mass spectrum: MALDI-TOF: m/z 1676.5.

Hydrogen NMR spectrum: ¹H NMR (600 MHz, CDCl₃): 8.65-8.64 (d, 2H), 7.28-7.23 (t, 2H), 3.95-3.94 (d, 4H), 1.98-1.94 (t, 4H), 1.341.26 (m, 40H), 0.12-0.09 (m, 84H).

Carbon NMR spectrum: ¹³C NMR (150 MHz, CDCl₃): 161.56, 139.43, 135.31, 131.43, 131.18, 118.87, 107.88, 46.37, 38.01, 33.70, 31.14, 29.69, 25.99, 23.11, 17.62, 1.86.

S9. A preparation method of a compound represented by formula A1 was the same as that of the compound represented by formula A in step S1, where a reactant was 10-bromo-1-decene (A1; t₃=t₄=8).

S10. A preparation method of a compound represented by formula B1 was the same as that of the compound represented by formula B in step S2, where a reactant was 2,2-bis(9-enyl-decane)dimethyl malonate. The structural characterization data was as follows:

Mass spectrum: MS (EI-MS): m/z 350.

Hydrogen NMR spectrum: ¹H NMR (300 MHz, CDCl₃): 5.855.72 (m, 2H), 4.944.89 (m, 4H), 3.65 (s, 6H), 2.032.01 (d, 4H), 1.421.24 (m, 32H).

Carbon NMR spectrum: ¹³C NMR (75 MHz, CDCl₃): 177.11, 139.13, 114.18, 114.12, 67.71, 51.22, 45.69, 33.87, 32.48, 29.50, 29.38, 29.07, 28.89, 27.64, 27.43.

511, a compound shown in formula C1 was prepared according to the following method:

under nitrogen protection, 12.7 g (36.29 mmol) of 2-(9-enyl-decane)dodecyl-11-alkenoic acid methyl ester, 100 mL of water-free n-hexane, and 27 g (78 mmol) of perfluoroiodobutane (t₅=t₆=3) were successively added to a 250 mL there-necked flask; under the liquid nitrogen environment, nitrogen replacement and heating to room temperature were conducted, and the above operations were repeated three times; a mixture was lowered to 0° C., 1 g (0.87 mmol) of tetrakis(triphenylphosphine)palladium was added, and a reaction was conducted at room temperature for 48 h; a product was filtered through a dry silica gel column and spin-dried; under nitrogen conditions, 4.14 g (109 mmol) of lithium aluminum hydride and 50 mL of water-free ether were added to another single-necked bottle; the spin-dried filtrate obtained in the previous step was added dropwise to 20 mL of the ether, at a speed that the solution was refluxed, and a reflux reaction was conducted for 6 h; a product was cooled to room temperature, water and 30% sulfuric acid were slowly added, and extracted with ether; an obtained product was subjected to washing with an aqueous solution of sodium thiosulfate, washing with water, washing with saline, drying, spin-drying, and purifying with dichloromethane/petroleum ether (2/1, V/V) through a chromatographic column to obtain 14.9 g of a colorless liquid product, with a yield of 67%. Product structural characterization data was as follows:

Hydrogen NMR spectrum: ¹H NMR (300 MHz, CDCl₃): 3.55-3.54 (d, 2H), 2.101.95 (m, 4H), 1.59 (s, 4H), 1.451.27 (m, 33H).

Carbon NMR spectrum: ¹³C NMR (75 MHz, CDCl₃): 122.08, 121.66, 119.76, 118.88, 118.30, 117.91, 115.50, 114.93, 114.04, 110.53, 109.03, 108.50, 65.67, 40.51, 31.08, 30.80, 30.47, 29.56, 29.51, 29.15, 29.08, 26.89, 20.07, 19.97.

S12, a compound shown in formula D1 was prepared according to the following method:

in the dark, 11 g (15 mmol) of 13,13,14,14,15,15,16,16,16-nonafluoro-2-(11,11,12,12, 13,13,14,14,14-nonafluorotetradecyl)hexadecan-1-ol and 100 mL of dichloromethane were added to a 500 mL single-necked bottle and stirred; 4.72 g (18 mmol) of triphenylphosphine and 1.23 g (18 mmol) of imidazole were sequentially added, and 2.76 g (17.25 mmol) of iodine was added in batches under ice bath; a mixture was reacted overnight at room temperature; a product was spin-dried, filtered, washed with water, dried, and spin-dried to obtain 12.2 g of a colorless liquid product with a yield of 96%, which could be directly used in the next step.

S13, a compound shown in formula E1 was prepared according to the following method:

under nitrogen protection, 1.2 g (4 mmol) of 3,6-bis(thiophen-2-yl)-2,5-dihydropyrrolo[3,4-c]pyrrole-1,4-dione and 1.66 g (12 mmol) of potassium carbonate were added sequentially to a 250 mL three-necked flask; 100 mL of a N,N-dimethylformamide (DMF) solvent was added and stirred, heated to 110° C. and stirred for 1 h; 6.55 g (8.8 mmol) of 1,1,1,2,2,3,3,4,4,26,26,27,27,28,28,29,29,29-octadecafluoro-15-(iodomethyl)nonacosane (D1; t₃=t₄=8; t₅=t₆=3) was added, heated to 120° C. overnight; a mixture was cooled to room temperature, the potassium carbonate was removed by suction filtration, spin-dried, and purified with dichloromethane/petroleum ether (1/1, V/V) by a chromatographic column to obtain 1.96 g of a red powdery solid, with a yield of 27%.

Product structural characterization data was as follows:

Mass spectrum: MALDI-TOF: m/z 1789.6.

Hydrogen NMR spectrum: ¹H NMR (300 MHz, CD₂Cl₂): 8.888.87 (d, 2H), 7.707.68 (d, 2H), 7.327.29 (t, 2H), 4.044.02 (d, 4H), 2.142.06 (m, 8H), 1.90 (s, 2H), 1.57 (s, 22H), 1.301.25 (d, 50H).

Carbon NMR spectrum: ¹³C NMR (75 MHz, CD₂Cl₂): 161.56, 140.17, 134.85, 130.52, 129.98, 128.17, 107.94, 46.04, 37.74, 31.13, 30.69, 30.38, 29.92, 29.46, 26.15, 20.01.

S14, a compound shown in formula M2 was prepared according to the following method:

under nitrogen protection in the dark, 100 mL of chloroform and 0.895 g (0.5 mmol) of 2,5-bis(13,13,14,14,15,15,16,16,16-nonafluoro-2-(11,11,12,12,13,13,14,14,14-nonafluorotetradecyl)hexadecyl)-3,6-bis(thiophene-2-yl)-2,5-dihydropyrrolo[3,4-c]pyrrole-1,4-dione (E1) were added to a 250 mL three-necked flask, 196 mg (1.1 mmol) of N-bromosuccinimide (NBS) was added in batches, and stirred at room temperature for 3 h; a mixture was extracted with dichloromethane, organic phases were combined, dried over magnesium sulfate, and spin-dried; a product was purified with dichloromethane/petroleum ether (1/1, V/V) by a chromatographic column to obtain 0.51 g of a red powdery solid, with a yield of 52%.

Product structural characterization data was as follows:

Mass spectrum: MALDI-TOF: m/z 1946.2.

Hydrogen NMR spectrum: ¹H NMR (300 MHz, CD₂Cl₂): 8.658.63 (d, 2H), 7.297.27 (d, 2H), 3.953.93 (d, 4H), 2.102.07 (m, 8H), 1.85 (s, 2H), 1.57 (s, 16H), 1.30 (s, 56H).

Carbon NMR spectrum: ¹³C NMR (75 MHz, CD₂Cl₂): 161.39, 139.38, 135.31, 131.44, 131.15, 118.72, 107.99, 46.31, 37.76, 31.12, 30.75, 30.45, 29.95, 29.37, 29.23, 29.10, 26.15, 20.05.

S15, a terpolymer shown in formula I was prepared according to the following method:

0.167 g of a monomer represented by formula M1 (0.1 mmol), 0.583 g of a monomer represented by formula M2 (0.3 mmol), and a monomer represented by formula M3, 5,5-bistrimethylsilyl-2,2′-bithiophene (BT) (0.4 mmol) with 4 mL of water-free chlorobenzene were added to a 50 mL Schlenk bottle, a resulting reaction system was cooled by liquid nitrogen and subjected to nitrogen replacement three times; 4.04 mg of tris(dibenzylideneacetone)dipalladium (0.0044 mmol) and 5.5 mg of o-trimethylphosphine (0.018 mmol) were added; a reaction was conducted by reflux stirring at 115° C. for 48 h, terminated, and 2 mL of bromobenzene was added to react overnight, to complete end-capping of the polymer; a reaction mixture was cooled to room temperature, and poured into 200 mL of a methanol solution containing 15 mL of hydrochloric acid for sedimentation, and then suction filtration was conducted to collect a black solid; the product was purified and separated by a Soxhlet extractor, where washing solvents were methanol (12 h), acetone (12 h), n-hexane (12 h), and chloroform (24 h) in sequence; the solution was extracted after the chloroform was spin-dried, to obtain 0.511 g of a blue-black lumpy polymer solid.

Product characterization data was as follows:

The molecular weight characterization data of the polymer were as follows: a weight average molecular weight was 148,909, a number average molecular weight was 71,488, and a molecular weight distribution index was 2.08.

As was seen from the data, the purple-black polymer solid product had a correct structure, which was the polymer shown in formula I; R₁ was 6-ethyl-1,11-bis(1,1,1,3,5,5,5-heptamethyltrisiloxane) (t₁=t₂=5); R₂ was 13,13,14,14,15,15,16,16,16-nonafluoro-2-(11,11,12,12,13,13,14,14,14-nonafluorotetradecyl)hexadecyl (t₃=t₄=8, t₆=t₆=3); n was an integer of 15 to 30.

The determination results of spectral properties, electrochemical properties, thermodynamic properties, and field effect transistor properties in the terpolymers prepared by the above examples were as follows:

1) Spectral Properties of the Terpolymers

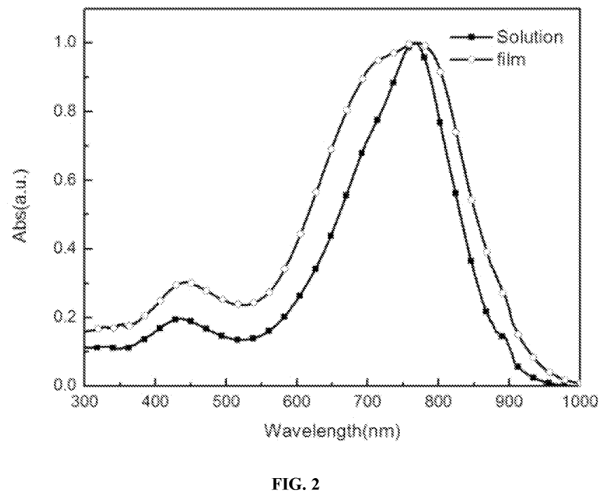

FIG. 2 showed a UV-Vis absorption spectrum of the terpolymers in a chloroform solution and a polymer film on a quartz plate. It was seen from FIG. 2 that a maximum absorption sideband peak of the terpolymer in the quartz plate was about 1000 nm, and a corresponding optical band gap was 1.24 eV (the optical band gap was calculated according to an equation E_{g}=1240/λ, where E_{g} was the optical band. gap, which was a boundary value of the UV absorption curve).

2) Electrochemical Properties of the Terpolymers

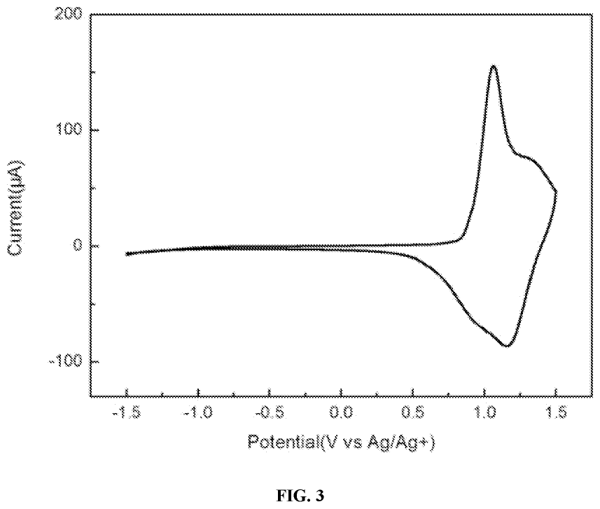

FIG. 3 showed a cyclic voltammetry curve of the terpolymer. The test was conducted by a three-electrode system: a working electrode was a platinum electrode coated with the terpolymer film, a platinum wire was a counter electrode, Ag/AgCl was a reference electrode, and Bu4NPF6 was used as a supporting electrolyte. The test conditions were as follows: a scanning range was −1.5 V to 1.5 V (vs. Ag/AgCl), and a scanning rate was 100 mV/s.

Electrochemical tests showed that the terpolymer had an initial oxidation potential of around 0.80 V, and a highest occupied molecular orbital (HOMO) energy level calculated therefrom was −5.20 eV, indicating that the terpolymer had a high oxidative stability and a desirable hole injection ability.

3) Thermodynamic Properties of the Terpolymers

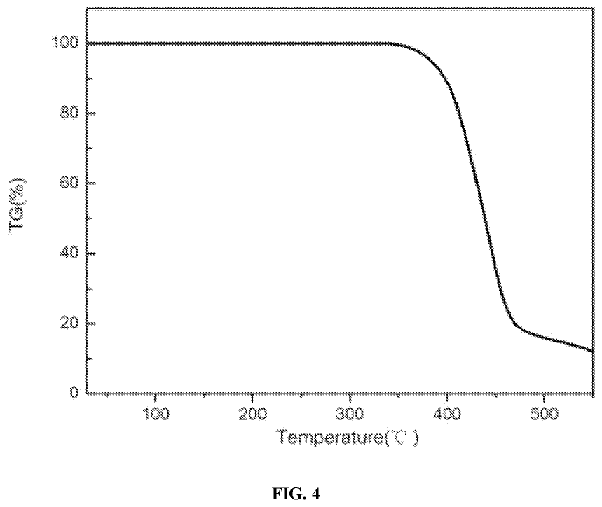

FIG. 4 is a TGA curve of the terpolymer. It was seen from FIG. 4 that 5% thermal weight loss had a decomposition temperature of around 380° C., indicating that the terpolymer had an excellent thermal stability.

4) Field Effect Transistor Properties of the Terpolymers

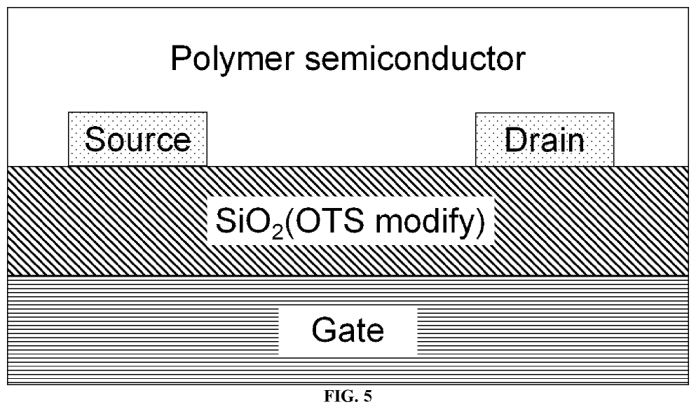

FIG. 5 showed a schematic structural diagram of an organic field effect transistor. As shown in FIG. 5, a bottom gate bottom contact (BGBC)-based field effect transistor device was used, a highly-doped silicon wafer was used as a substrate, octadecyltrichlorosilane-modified silicon dioxide (300 nm) was used as an insulating layer, and source electrode S (source) and drain electrode D (drain) each were made of gold (Au). An organic semiconductor layer (polymer semiconductor) composed of the terpolymer represented by formula I was prepared by spin-coating a terpolymer solution of 10 mg/mL o-dichlorobenzene, and the polymer film was subjected to annealing.

The electrical properties of the organic field effect transistors (OTFTs) were measured with a Keithley 4200SCS semiconductor tester at room temperature and in air. Two key parameters that determine the performance of OFETs are mobility (μ) and on/off ratio (Ion/Ioff). Mobility refers to an average drift velocity of carriers (cm²/V·s) under a unit electric field, reflecting the mobility of holes or electrons in a semiconductor under an electric field. The on/off ratio is defined as a ratio of currents of a transistor in an “on” state and an “off” state under a certain gate voltage, reflecting an on/off performance of the device. A high-performance field effect transistor should have the highest possible mobility and on/off ratio.

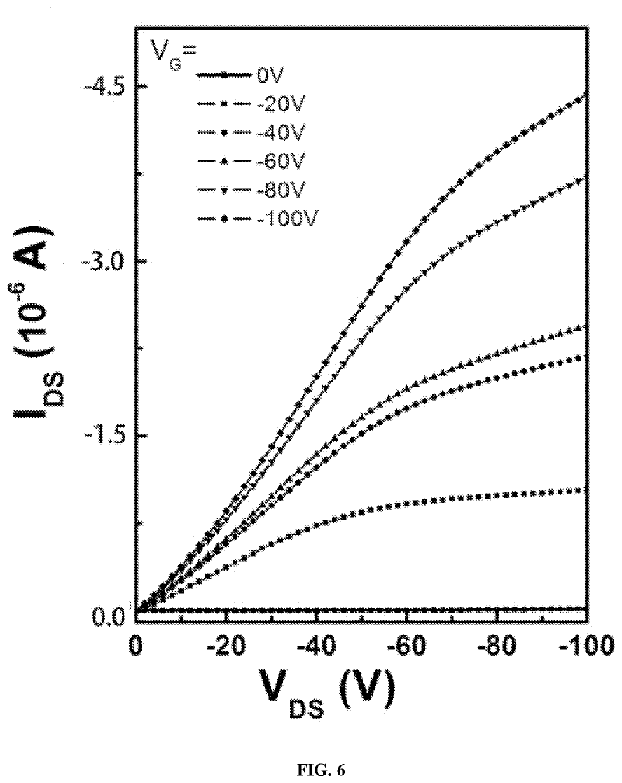

FIG. 6 was an output characteristic curve of the prepared field effect transistor under different gate voltages V_{G} when an annealing temperature was 150° C. The results show a desirable linear region and saturation region, indicating that OTFTs fabricated from the terpolymers had a desirable field-effect control performance.

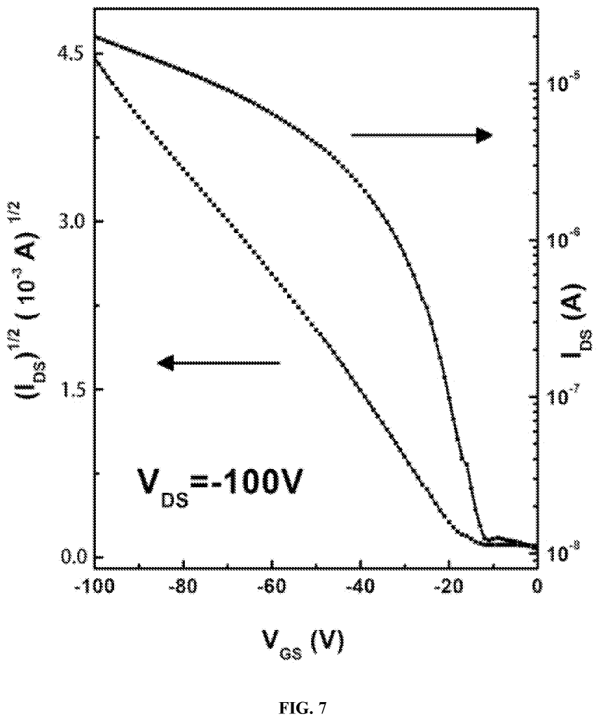

FIG. 7 showed a transfer characteristic curve of the fabricated field effect transistor when the annealing temperature was 150° C. and a source-drain voltage was −100V. From the data in the figure, it was calculated that the field effect transistor had a mobility of 6.5×10⁻³ cm²/V·s.

A carrier mobility was calculated from an equation as follows:

I_{DS}=(W/2L)C_{iμ}(V_{g}−V_{T})₂ (saturation region, V_{DS}=V_{g}−V_{T})

I_{D}S was a drain current, μ was a carrier mobility, V_{G} was a gate voltage, V_{T} was a threshold voltage, W was a channel width (W=1400 m), L was a channel length (L=10 m), and Cᵢ was an insulator capacitance (Cᵢ=7.5×10⁻⁹ F/cm²). V_{G} was plotted with (ID_{S, sat})^{1/2}, and linear regression was conducted, the carrier mobility (μ) was calculated from a slope of the regression line, and V_{T} was obtained from an intersection of the regression line and the X-axis. Mobility was calculated from a slope of a transfer curve according to the formula. I_{DS}=(W/2L)C_{iμ}(V_{G}—V_{T})². The on/off ratio was derived from a ratio of a maximum value to a minimum value of the source-drain current on the right side in FIG. 7.

In conclusion, the above experimental results showed that the DPP-based terpolymer provided in this example was an excellent organic semiconductor material. The desirable device performance depended on the large π-plane skeleton and desirable solution processability of the material. In the present disclosure, not only the synthesis method is simple and effective, but also a series of DPP-based polymer materials can be prepared by changing different alkyl substituents and acceptor units (A). This is extremely helpful for studying a relationship between the structure and properties of the organic semiconductor materials, and can further guide the design and synthesis of high-performance polymer materials.

The present disclosure provides a terminal functional side chain-substituted DPP-based terpolymer and a preparation method and use thereof, which have the following beneficial effects compared with the prior art:

I, the DPP-based terpolymers with different substitutions have desirable solubility, a high solubility of the siloxy chain improves a solubility of the fluorine chain-modified polymer, which has an excellent solubility in conventional solvents.

II, the DPP-based terpolymers with different substitutions each are a linear acceptor-donor-acceptor (A-D-A) conjugated molecule with an alternating ADA configuration and a rigid large π-planar structure, which are expected to prepare high-mobility OTFTs devices.

III, the preparation method has simplicity and high efficiency, cheap raw materials, and low synthesis cost, and the polymerization method has high universality and desirable repeatability, which can be generalized and applied to the synthesis of other polymers containing various electron-deficient acceptor units (A).

IV, DPP-based terpolymers with different substitutions have lower highest occupied molecular orbital (HOMO) energy levels (about −5.20 eV), desirable anti-oxidation ability due to high stability to oxygen, and desirable matching with gold electrodes, which is beneficial to obtain high-mobility OTFTs devices.

V, the DPP-based terpolymers have a mobility (μ) of up to 6.5×10⁻³ cm²/V·s in the preparation of OTFTs in organic semiconductor layers, with desirable prospects for use in the OTFTs.

It can be understood that the above implementations are merely exemplary implementations used to illustrate the principle of the present disclosure, and the present disclosure is not limited thereto. Various modifications and improvements can be made by those of ordinary skill in the art without departing from the spirit and essence of the present disclosure, and these modifications and improvements are also considered as falling within the protection scope of the present disclosure.

What is claimed is:

1. A terminal functional side chain-substituted diketopyrrolopyrrole (DPP)-based terpolymer, having the following structural formula: 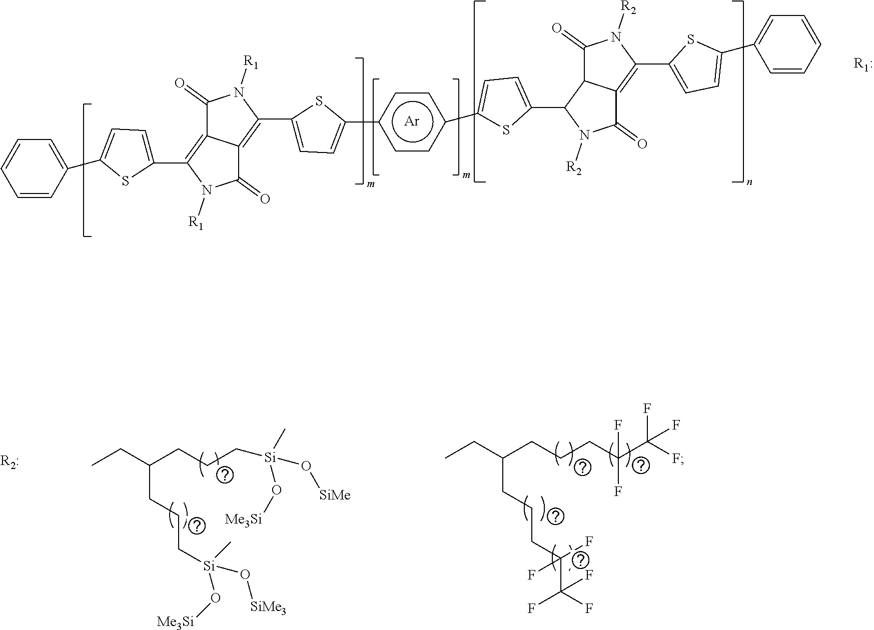
wherein
R₁ is a terminal siloxy-substituted swallow-tailed chain with 22 to 52 carbon atoms in total, and t₁ and t₂ are independently an integer of 1 to 18;
R₂ is a semifluoroalkyl-substituted swallow-tailed chain with 12 to 60 carbon atoms in total and 10 to 46 fluorine atoms in total, t₃ and t₄ are independently an integer of 1 to 16, and t₅ and t₆ are independently an integer of 1 to 10; and
Ar is selected from the group consisting of aryl, heteroaryl, substituent-containing aryl, and substituent-containing heteroaryl, and m and n are independently an integer of 5 to 100.

2. The terminal functional side chain-substituted DPP-based terpolymer according to claim 1, wherein in R₁, the branched alkyl with 22 to 52 carbon atoms in total is selected from the group consisting of 2-pentylheptyl, 2-hexyloctyl, 2-heptylnonyl, 2-octyldecyl, 2-nonylundecyl, and 2-decyldodecyl; and/or,
in R₂, the branched alkyl with 12 to 60 carbon atoms in total is selected from the group consisting of 4-undecylpentadecyl, 4-dodecylhexadecyl, and 4-tridecylheptadecyl, and the semifluoroalkyl-substituted fluorine chain with 10 to 46 fluorine atoms in total is selected from the group consisting of nonafluorobutyl, heptafluoropropyl, and pentafluoroethyl; and/or,
in Ar, the aryl is selected from the group consisting of monocyclic aryl, bicyclic aryl, and polycyclic aryl; and/or, the heteroaryl is selected from the group consisting of monocyclic heteroaryl, bicyclic heteroaryl, and polycyclic heteroaryl; and/or, in the substituent-containing aryl and the substituent-containing heteroaryl, the substituent is selected from the group consisting of C₁ to C₅₀ alkyl, C₁ to C₅₀ alkoxy, C_{I} to C₅₀ alkylthio, a nitrile group, and a halogen atom, and there are 1 to 4 of the substituents.

3. The terminal functional side chain-substituted DPP-based terpolymer according to claim 2, wherein R₁ is 10-ethyl-1,19-bis(1,1,1,3,5,5,5-heptamethyltrisiloxane)nonadecane; and/or,
R₂ is 15-ethyl-1,1,1,2,2,3,3,4,4,26,26,27,27,28,28,29,29,29-octadecafluorononacosane; and/or,
Ar is selected from the group consisting of 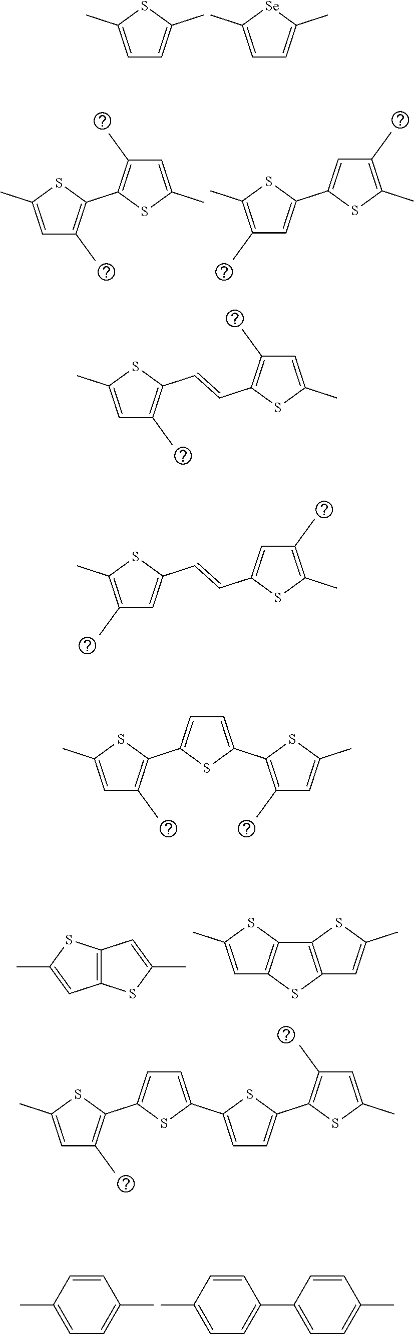 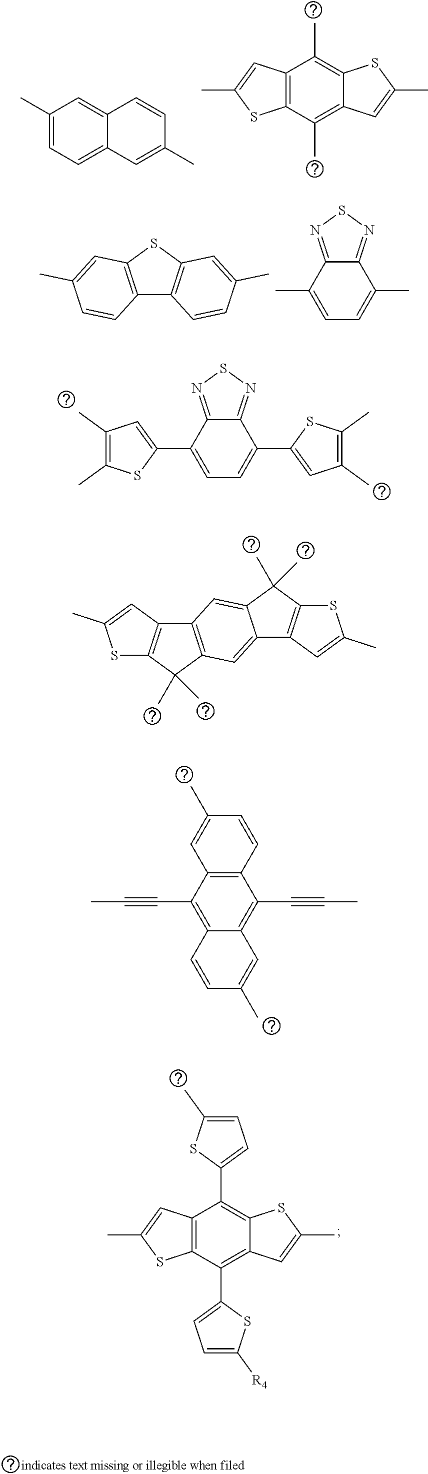
and
R₃ and R₄ are independently selected from the group consisting of hydrogen, C₁ to C₅₀ alkyl, C₁ to C₅₀ alkoxy, a nitrile group, and a halogen atom, and m and n are independently an integer of 5 to 50.

4. The terminal functional side chain-substituted DPP-based terpolymer according to claim 2, wherein heteroatoms in the monocyclic heteroaryl, the bicyclic heteroaryl, and the polycyclic heteroaryl are independently at least one of oxygen, sulfur, and selenium.

5. A preparation method of the terminal functional side chain-substituted DPP-based terpolymer according to claim 1, the method comprising the following steps:
in the presence of an inert gas, a palladium catalyst, and a phosphine ligand, mixing the following monomers represented by M1, M2, and M3 in an organic solvent to obtain a reaction system, and conducting a reaction to obtain the terpolymer; wherein
M1 is 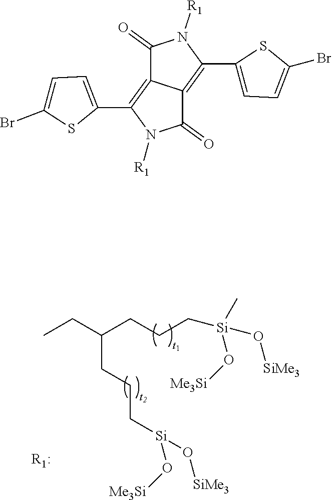
M2 is 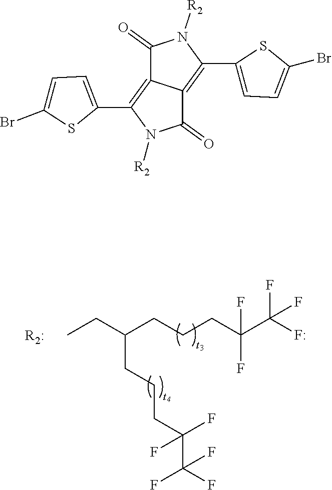
and
M3 is 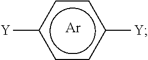
and Y is selected from the group consisting of a trialkyltin group and a borate group.

6. The method according to claim 5, wherein in R₁, the branched alkyl with 22 to 52 carbon atoms in total is selected from the group consisting of 2-pentylheptyl, 2-hexyloctyl, 2-heptylnonyl, 2-octyldecyl, 2-nonylundecyl, and 2-decyldodecyl; and/or,
in R₂, the branched alkyl with 12 to 60 carbon atoms in total is selected from the group consisting of 4-undecylpentadecyl, 4-dodecylhexadecyl, and 4-tridecylheptadecyl, and the semifluoroalkyl-substituted fluorine chain with 10 to 46 fluorine atoms in total is selected from the group consisting of nonafluorobutyl, heptafluoropropyl, and pentafluoroethyl; and/or,
in Ar, the aryl is selected from the group consisting of monocyclic aryl, bicyclic aryl, and polycyclic aryl; and/or, the heteroaryl is selected from the group consisting of monocyclic heteroaryl, bicyclic heteroaryl, and polycyclic heteroaryl; and/or, in the substituent-containing aryl and the substituent-containing heteroaryl, the substituent is selected from the group consisting of C1 to C₅₀ alkyl, C1 to C₅₀ alkoxy, C₁ to C₅₀ alkylthio, a nitrile group, and a halogen atom, and there are 1 to 4 of the substituents.

7. The method according to claim 6, wherein R₁ is 10-ethyl-1,19-bis(1,1,1,3,5,5,5-heptamethyltrisiloxane)nonadecane; and/or,
R₂ is 15-ethyl-1,1,1,2,2,3,3,4,4,26,26,27,27,28,28,29,29,29-octadecafluorononacosane; and/or,
Ar is selected from the group consisting of 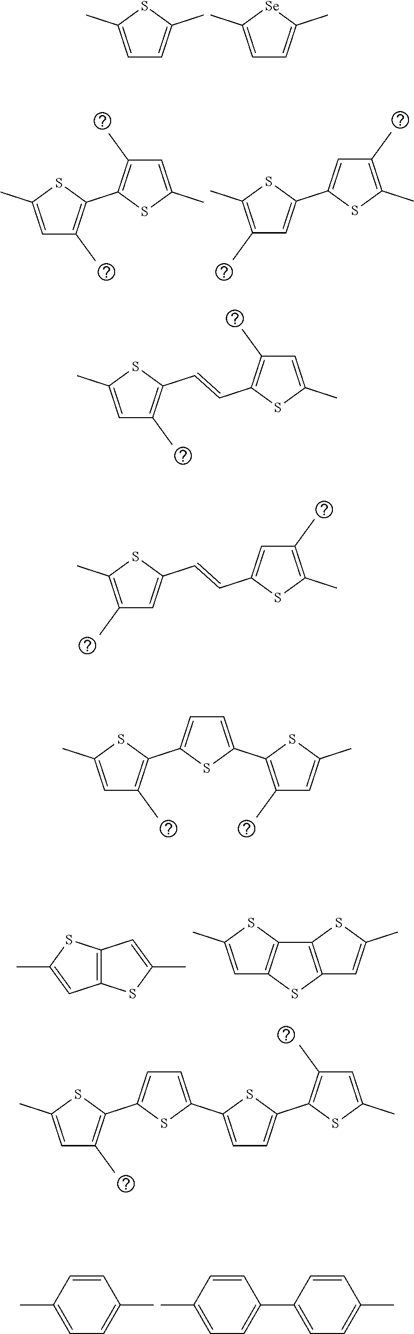 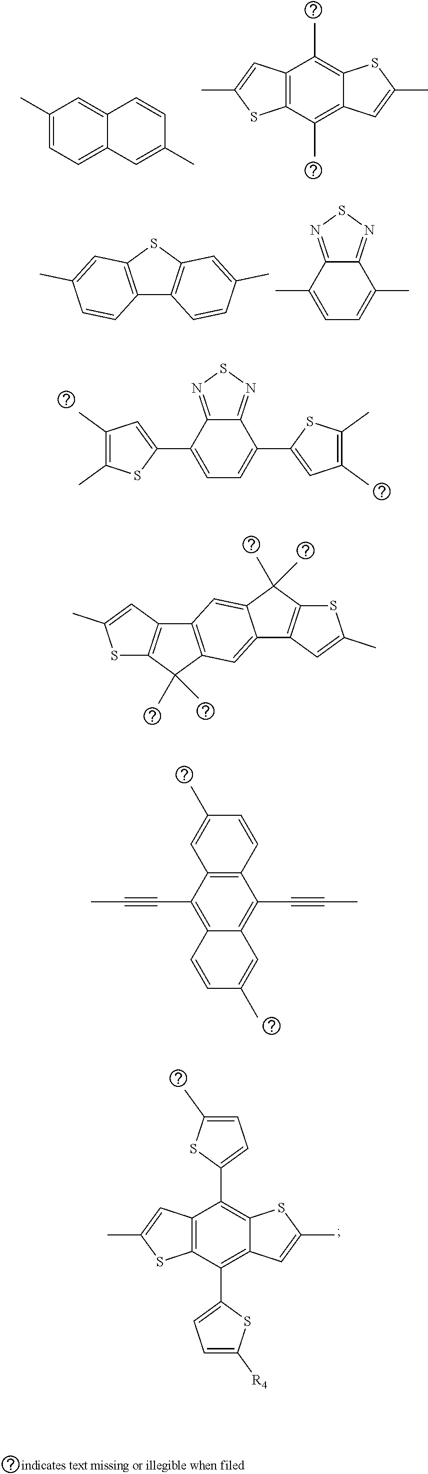
and
R₃ and R₄ are independently selected from the group consisting of hydrogen, C₁ to C₅₀ alkyl, C₁ to C₅₀ alkoxy, a nitrile group, and a halogen atom, and m and n are independently an integer of 5 to 50.

8. The method according to claim 6, wherein heteroatoms in the monocyclic heteroaryl, the bicyclic heteroaryl, and the polycyclic heteroaryl are independently at least one of oxygen, sulfur, and selenium.

9. The method according to claim 5, further comprising the following step after the reaction is completed: adding phenylboronic acid or bromobenzene to the reaction system to conduct a polymer end-capping treatment for 1 h to 24 h; wherein
the phenylboronic acid or the bromobenzene and the monomer represented by M1 have a molar dosage ratio of (10-100):1.

10. The method according to claim 6, further comprising the following step after the reaction is completed: adding phenylboronic acid or bromobenzene to the reaction system to conduct a polymer end-capping treatment for 1 h to 24 h; wherein
the phenylboronic acid or the bromobenzene and the monomer represented by M1 have a molar dosage ratio of (10-100):1.

11. The method according to claim 7, further comprising the following step after the reaction is completed: adding phenylboronic acid or bromobenzene to the reaction system to conduct a polymer end-capping treatment for 1 h to 24 h; wherein
the phenylboronic acid or the bromobenzene and the monomer represented by M1 have a molar dosage ratio of (10-100):1.

12. The method according to claim 8, further comprising the following step after the reaction is completed: adding phenylboronic acid or bromobenzene to the reaction system to conduct a polymer end-capping treatment for 1 h to 24 h; wherein
the phenylboronic acid or the bromobenzene and the monomer represented by M1 have a molar dosage ratio of (10-100):1.

13. The method according to claim 5, wherein the reaction is conducted at 100° C. to 130° C. for 24 h to 72 h.

14. The method according to claim 6, wherein the reaction is conducted at 100° C. to 130° C. for 24 h to 72 h.

15. The method according to claim 7, wherein the reaction is conducted at 100° C. to 130° C. for 24 h to 72 h.

16. The method according to claim 8, wherein the reaction is conducted at 100° C. to 130° C. for 24 h to 72 h.

17. The method according to claim 5, wherein the palladium catalyst, the phosphine ligand, and the monomer represented by M1 have a molar dosage ratio of (0.01-0.05):(0.09-0.12):1; and/or,
the monomer represented by M1, the monomer represented by M2, and the monomer represented by M3 have a molar dosage ratio of 1:(1-5.05):(2-6.05).

18. The method according to claim 6, wherein the palladium catalyst, the phosphine ligand, and the monomer represented by M1 have a molar dosage ratio of (0.01-0.05):(0.09-0.12):1; and/or,
the monomer represented by M1, the monomer represented by M2, and the monomer represented by M3 have a molar dosage ratio of 1:(1-5.05):(2-6.05).

19. The method according to claim 5, wherein the inert gas is selected from the group consisting of nitrogen and argon; and/or,
the palladium catalyst is at least one of tetrakis(triphenylphosphine)palladium, tris(tri-p-methylphenylphosphine)palladium, tris(dibenzylideneacetone)dipalladium, and [1,4-bis(diphenylphosphino)butane]palladium(II) dichloride; and/or,
the phosphine ligand is at least one of triphenylphosphine, o-trimethylphosphine, tris(2-furyl)phosphine, and 2-(di-tert-butylphosphine)biphenyl; and/or,
the organic solvent is at least one of toluene, chlorobenzene, and N,N-dimethylformamide; and/or,
the trialkyltin group is selected from the group consisting of trimethyltin and tributyltin; and/or,
the borate group is selected from the group consisting of 1,3,2-dioxaborolane-2-yl and 4,4,5,5-tetramethyl-1,2,3-dioxaborolane-2-yl.

20. A preparing method of any one of an organic light-emitting diode, a field effect transistor, a flexible active matrix display, an organic radio frequency electronic trademark, an organic sensor/memory, an organic functional plastic, an electronic paper, and a solar cell by using the terminal functional side chain-substituted DPP-based terpolymer according to claim 1.
